(19) United States
(12) Patent Application Publication
(10) Pub. No.: US 2022/0002359 A1
(43) Pub. Date: Jan. 6, 2022

(54) Voltage-Gated Calcium Channel Auxilliary Subunit Alpha 2 Delta and Uses Thereof

(71) Applicant: Novassay, S.A., Epalinges (CH)

(72) Inventors: Franz Kricek, Bidemannsdorf (AT); George Skouteris, Epalinges (CH)

(73) Assignee: Novassay S.A., Epalinges (CH)

(21) Appl. No.: 16/616,752

(22) PCT Filed: May 19, 2018
(86) PCT No.: PCT/EP2018/063245

§ 371 Date: Nov. 25, 2019

Related U.S. Application Data
(60) Provisional application No. 62/511,774, filed on May 26, 2017.

(51) Int. Cl. C07K 14/47; C07K 16/18; G01N 33/68; A61K 31/197

(52) U.S. Cl. CPC C07K 14/47; C07K 16/18; G01N 2500/04; A61K 31/197; G01N 33/6845

(57) ABSTRACT

The Voltage-Gated Calcium Channel auxiliary subunit α2δ-1 is the target/receptor of gabapentinoid compounds known to exert therapeutic effects as for example in Epilepsy and Neuropathic pain. Gabapentinoids are known to exert their action via binding Arginine (R) within an RRR motif located at the N-terminal of the α2δ-1. The present invention describes a novel binding site for gabapentinoids which is located within the VGCC_a2 domain and within an IKAK aminoacid sequence of the α2δ-1. Such newly identified amino acid binding site finds utility in the identification and characterization of novel compounds with therapeutic properties in Neuropathic Pain and in other disorders and conditions in which α2δ-1 is involved in.

The present invention relates to voltage-gated calcium channels (VGCCs), and particularly, although not exclusively to, novel peptides located within the α2δ-1 subunit of voltage-gated calcium channels, which form a binding site, for example, for a gabapentinoid. The invention also extends to kits comprising the isolated peptide, and to methods of identifying agents that bind to the peptide. The invention also encompasses methods of preparing the peptide, agents identified using the peptide, antibodies capable of binding to the isolated peptide, animals expressing a mutated version of the peptide and uses thereof.

### BACKGROUND OF THE INVENTION

Voltage-gated calcium (CaV) channels are essential components for many key functions in excitable cells, including neurotransmitter release and muscle contraction. The α1 subunit was found to bind the calcium channel blockers and was identified to be the pore-forming channel subunit. The β and α2δ subunits were then termed auxiliary or accessory subunits [1].

Four α2δ subunit genes have been identified. CACNA2D1 encodes α2δ-1, which was first identified in skeletal muscle. It is present in cardiac and smooth muscle and in the brain. The CACNA2D2 and CACNA2D3 genes encoding α2δ-2 and α2δ-3, are differentially expressed in neurons and in some other tissues [2]. CACNA2D4, encoding α2δ-4, has shown expression which is mainly non-neuronal [3].

The α2δ-1 subunit contains certain domains, including a Von Willebrand Factor A (VWF-A or VW A) domain which is known to be involved in binding to a number of cell adhesion and extracellular matrix proteins (SEQ ID NO. 1). The VWA domains are involved in protein protein interactions, via their metal ion-dependent adhesion site (MIDAS) motif. There are also two bacterial chemosensory-like or Cache domains in α2δ subunits, situated downstream of the VWA domain [4].

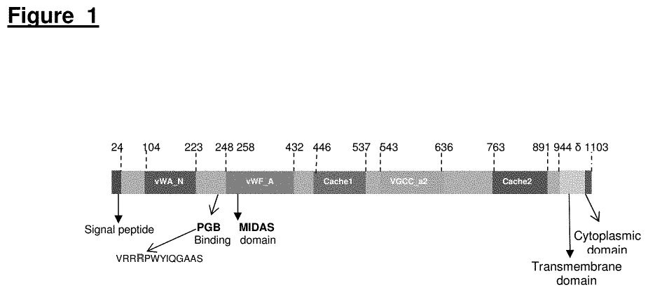

The approximate positions of the VWA_N and VWF-A domains, the two bacterial chemosensory domains (CACHE1 and CACHE2) and the Voltage-Gated Calcium Channel α2 domain (VGCC_α2) are illustrated in FIG. 1.

The α2δ subunits and their binding ligands have been considered as therapeutic targets in neuropathic pain, in epilepsy [1,5,10] and a number of diseases and conditions to include lower urinary tract symptoms (WO2004054560 A1), obstructive pulmonary disease (WO2004054577 A1), sexual dysfunction (U.S. Pat. No. 7,432,299 B2) and Kawasaki disease (EP 2 116 618 A1).

In addition, targeted disruption of the α2δ-1 subunit in a knockout mouse is not lethal, with these animals lacking high-affinity gabapentin binding sites demonstrating a significantly decreased basal myocardial contractility and relaxation and a decreased L-type Ca current peak current amplitude [9].

The α2δ-1 subunit has also been implicated as a therapeutic target in cancer [16, WO 2012/113266] and in the modulation of synaptogenesis [17, US2011104181 A1].

It is well-established that α2δ-1 plays a central role in the development of chronic pain associated with nerve injury (neuropathic pain) [1, 5]. Experimental peripheral nerve injury results in elevated α2δ-1 mRNA levels in the damaged sensory neurons (trigeminal neurons and DRGs), as well as corresponding increase of α2δ-1 protein in DRGs and spinal cord [6,7].

The role of the α2δ-1 subunit in pain has been investigated further using transgenic animal (mouse) models. It has been shown that over-expression of α2δ-1 exclusively in neuronal tissues in transgenic mice has revealed an injury-unrelated neuropathic phenotype of hyperalgesia and tactile allodynia. This clearly establishes α2δ-1 as a subunit involved in the development of neuropathic pain phenotype [8]. Naive α₂δ-1^{-/-} mice have shown a marked behavioural deficit in mechanical and cold sensitivity, but no change in thermal nociception threshold. The lower mechanical sensitivity was mirrored by a reduced in vivo electrophysiological response of dorsal horn wide dynamic range neurons. The Ca_{V}2.2 level is reduced in brain and spinal cord synaptosomes from α₂δ-1^{-/-}. The ability of the anti-epileptic drug pregabalin to alleviate mechanical hypersensitivity is lost in partial sciatic ligation neuropathy α2δ-1^{-/-} mice. It was then conclusively shown that α2δ-1 is essential for rapid development of mechanical hypersensitivity in this nerve injury model of neuropathic pain [9,13].

Pregabalin (PGB) is a synthetic branched chain γ-amino butyric acid (GABA) with analgesic, anticonvulsant and anxiolytic activities, and has shown potent and selective binding to the voltage-gated calcium channel subunits α2δ-1 and α2δ-2 [10, WO2008004067 A2].

Gabapentin and pregabalin are anti-epileptic drugs which also have therapeutic use in neuropathic pain (i.e. display analgesic activity) [10]. Both compounds bind to α2δ-1 and α2δ-2, on amino acids positioned in an RRR motif located N-terminal to the VWA domain [11,12]. They specifically bind to Arginine (R) at position 217 (i.e. position 241 in SEQ ID NO. 1, which corresponds to the protein sequence of the mature α2δ-1 protein). Binding of gabapentinoids on their α2δ-1 receptor is required for their analgesic effect, as shown in the prior art [1]. Binding of Pregabalin revealed an IC₅₀ of 23 nM whereas binding of NVA1309 revealed an IC₅₀ of 2.1 nM, using recombinant membranes over-expressing the human voltage-gated calcium channel subunit α2δ-1 (PCT/EP2014/077937). In a knock-in mutant mouse bearing a mutation in the RRR motif of α2δ-1 (RRR mutated to RRA), pregabalin was no longer effective in alleviating chronic pain resulting from nerve injury [13, WO2004089071 A1]. These data further confirmed α2δ-1 as the protein target responsible for the therapeutic effects of gabapentinoid drugs in neuropathic pain.

Thus, it is well known that gabapentinoids are effective at treating a variety of conditions. However, considering their effectiveness, very few gabapentinoids, which can be used clinically, have been identified or developed.

There is therefore a need for new methods of identifying agents that alter the function of voltage-gated calcium channels by binding to α2δ-1 subunits.

### DESCRIPTION OF THE INVENTION

Thus, according to a first aspect of the invention, there is provided an isolated α2δ-1 peptide consisting of a fragment of an amino acid sequence substantially as set out in SEQ ID No. 5 and encompassing an amino acid sequence substantially as set out in SEQ ID No. 20.

Like the known RRR motif, which is responsible for binding the gabapentinoids, gabapentin and pregabalin, the peptide according to the first aspect is derived from the VGCC_α2 domain of the α2δ-1 subunit of voltage-gated calcium channels. The RRR motif is distinct from the peptide according to the first aspect; in situ, the RRR motif is located upstream of the VWA domain of the mature α2δ-1 protein (see FIGS. 1 & 2). Thus, the region of the α2δ-1 subunit that corresponds to the peptide according to the first aspect is capable of being bound by certain gabapentinoids independently of the RRR motif (see the Examples). Therefore, the peptide according to the first aspect may be used to identify new agents that bind to the α2δ-1 subunit. These agents may be gabapentinoids or non-gabapentinoids, and they may be used to treat conditions in which the α2δ-1 subunit of voltage-gated calcium channels plays a role.

The α2δ-1 subunit is an auxiliary or accessory subunit of voltage-gated calcium channels. It is referred to as an auxiliary or accessory subunit because, unlike the α1 subunit of voltage-gated calcium channels, it is not one of the pore-forming subunits of such voltage-gated channels. In one embodiment, the human α2δ-1 subunit comprises a sequence referred to herein as SEQ ID No. 1, as follows:

MAAGC
MAAGCLLALTLTLFQSLLIGPSSEEPFPSAVTIKSWVDKMQEDLVTLAKT
ASGVNQLVDIYEKYQDLYTVEPNNARQLVEIAARDIEKLLSNRSKALVRL
ALEAEKVQAAHQWREDFASNEVVYYNAKDDLDPEKNDSPEGSQRIKPVFI
EDANFGRQISYQHAAVHIPTDIYEGSTIVLNELNWTSALDEVFKKNREED
PSLLWQVFGSATGLARYYPASPWVDNSRTPNKIKLYDVRRRPWYIQGAAS
PKDMLILVDVSGSVSGLTLKLIRTSVSEMLETLSDDDFVNVASFNSNAQD
VSCFQHLVQANVRNKKVLKDAVNNITAKGITDYKKGFSFAFEQLLNYNVS
RANCNKIIMLFTDGGEERAQEIFNKYNKDKKVRVFTFSVGQHNYDRGPIQ
WMACENKGYYYEIPSIGAIRINTQEYLDVLGRPMVLAGDKAKQVQWTNVY
LDALELGLVITGTLPVFNITGQFENKTNLKNQLILGVMGVDVSLEDIKRL
TPRFTLCPNGYYFAIDPNGYVLLHPNLQPKPIGVGIPTINLRKRRPNIQN
PKSQEPVTLDFLDAELENDIKVEIRNKMIDGESGEKTFRTLVKSQDERYI
DKGNRTYTWTPVNGTDYSLALVLPTYSFYYIKAKLEETITQARYSETLKP
DNFEESGYTFIAPRDYCNDLKISDNNTEFLLNFNEFIDRKTPNNPSCNAD
LINRVLLDAGFTNELVQNYWSKQKNIKGVKARFVVTDGGITRVYPKEAGE
NWQENPETYEDSFYKRSLDNDNYVFTAPYFNKSGPGAYESGIMVSKAVEI
YIQGKLLKPAVVGIKIDVNSWIENFTKTSIRDPCAGPVCDCKRNSDVMDC
VILDDGGFLLMANHDDYTNQIGRFFGEIDPSLMRHLVNISVYAFNKSYDY
QSVCEPGAAPKQGAGHRSAYVPSVADILQIGWWATAAAWSILQQFLLSLT
FPRLLEAVEMEDDDFTASLSKQSCITEQTQYFFDNDSKSFSGVLDCGNCS
RIFHGEKLMNTNLIFIMVESKGTCPCDTRLLIQAEQTSDGPNPCDMVKQP
RYRKGPDVCFDNNVLEDYTDCGGVSGLNPSLWYIIGIQFLLLWLVSGSTH
RLL
RLL

The isolated α2δ-1 peptide according to the first aspect may comprise a fragment of an amino acid sequence referred to herein as SEQ ID No. 5, as follows:

QPKP
QPKPIGVGIPTINLRKRRPNIQNPKSQEPVTLDFLDAELENDIKVEIRNK
MIDGESGEKTFRTLVKSQDERYIDKGNRTYTWTPVNGTDYSLALVLPTYS
FYYIKAKLEETITQARYSETLKPDNFEESGYTFIAPRDYCN
YCN

The amino acid sequence of SEQ ID No. 5 is a fragment of the α2δ-1 subunit according to SEQ ID No. 1.

In another embodiment, the fragment may comprise or consist of an amino acid sequence referred to herein as SEQ ID No. 20, as follows:

[SEQ ID No. 20]
IKAK

The amino acid sequence of SEQ ID No. 5 encompasses the amino acid sequence of SEQ ID No. 20. Thus, the amino acid sequence of SEQ ID No. 5 comprises or consists of the amino acid sequence of SEQ ID No. 20 and further includes N-terminal and C-terminal amino acids. Thus, in another embodiment, the fragment may comprise or consist of an amino acid sequence referred to herein as SEQ ID No. 20, and further include at least 1, 2, 3, 4, 5, 6, 7, 8, 9 or 10 N-terminal and/or C-terminal amino acids, which correspond to at least 1, 2, 3, 4, 5, 6, 7, 8, 9 or 10 amino acids that are located at the N-terminus and/or C-terminus of the fragment equivalent to SEQ ID No. 20 as disposed within SEQ ID No. 5.

In another embodiment, the fragment may comprise or consist of an amino acid sequence referred to herein as SEQ ID No. 21, as follows:

[SEQ ID No. 21]
IKAKLE

In another embodiment, the fragment may comprise or consist of an amino acid sequence referred to herein as SEQ ID No. 22, as follows:

[SEQ ID No. 22]
IKAKLEET

In another embodiment, the fragment may comprise or consist of an amino acid sequence referred to herein as SEQ ID No. 23, as follows:

[SEQ ID No. 23]
IKAKLEETITQA

The α2δ-1 peptide may comprise or consist of an amino acid sequence substantially as set out in any one of SEQ ID Nos. 20 to 23. Preferably, the first four amino acids of SEQ ID Nos. 20 to 23 are not mutated, altered or substituted. Most preferably, the fourth amino acid (i.e. K (lysine)) of SEQ ID No. 20, SEQ ID No. 21, SEQ ID No. 22 or SEQ ID No. 23 is not mutated, altered or substituted.

The RRR motif is a known binding site for the gabapentinoids, gabapentin and pregabalin. Thus, in another embodiment, the peptide according to the first aspect may also encompass a RRR motif or be conjugated to a separate peptide encompassing a RRR motif (SEQ ID No. 25).

According to a second aspect, there is provided an antibody or antigen-binding fragment thereof capable of binding or interacting with a peptide according to the first aspect or a peptide referred to in any of the following aspects.

The antibody may be a whole antibody (i.e. an immunoglobulin) with immunospecificity for the isolated α2δ-1 peptide according to the first aspect or the peptide referred to in any of the following aspects, preferably the antibody is an extracellular domain thereof or a functional fragment thereof. Such antibody fragments retain at least one antigen binding region of the corresponding full-length antibody. The antibody or fragment thereof may be a humanised antibody or fragment thereof. The antibody or functional fragment thereof may comprise a human monoclonal or polyclonal antibody or functional fragment thereof. The antibody fragment may be a VL fragment, a VH fragment, a Fd fragment, a Fd fragment, a Fv fragment, a Fab fragment, a Fab′ fragment or a F(ab′)₂ fragment. The antibody may be a single chain Fv (scFv) or a bispecific antibody (BsAb).

The antibody or functional fragment may be monovalent, divalent or polyvalent. Monovalent antibodies are dimers (HL) comprising a heavy (H) chain associated by a disulphide bridge with a light chain (L). Divalent antibodies are tetramer (H2L2) comprising two dimers associated by at least one disulphide bridge. Polyvalent antibodies may also be produced, for example by linking multiple dimers. The basic structure of an antibody molecule consists of two identical light chains and two identical heavy chains which associate non-covalently and can be linked by disulphide bonds. Each heavy and light chain contains an amino-terminal variable region of about 110 amino acids, and constant sequences in the remainder of the chain. The variable region includes several hypervariable regions, or Complementarity Determining Regions (CDRs), that form the antigen-binding site of the antibody molecule and determine its specificity for the antigen, i.e. the peptide according to the first aspect. On either side of the CDRs of the heavy and light chains is a framework region, a relatively conserved sequence of amino acids that anchors and orients the CDRs. Antibody fragments may include a bi-specific antibody (BsAb) or a chimeric antigen receptor (CAR).

As used herein, the term “human or humanised antibody” can mean an antibody, such as a monoclonal antibody, which comprises substantially the same heavy and light chain CDR amino acid sequences as found in a particular human antibody exhibiting immunospecificity for the α2δ-1 peptide. An amino acid sequence, which is substantially the same as a heavy or light chain CDR, exhibits a considerable amount of sequence identity when compared to a reference sequence. Such identity is definitively known or recognizable as representing the amino acid sequence of the particular human antibody. Substantially the same heavy and light chain CDR amino acid sequence can have, for example, minor modifications or conservative substitutions of amino acids. Such a human antibody maintains its function of selectively binding to the α2δ-1 recombinant peptide.

The term “human monoclonal antibody” can include a monoclonal antibody with substantially or entirely human CDR amino acid sequences produced, for example by recombinant methods such as production by a phage library, by lymphocytes or by hybridoma cells.

The antibody may be a recombinant antibody. The term “recombinant human antibody” can include a human antibody produced using recombinant DNA technology.

The term “antigen binding region” can mean a region of the antibody having specific binding affinity for its target antigen, for example, the α2δ-1 peptide, or an epitope thereof. The binding region may be a hypervariable CDR or a functional portion thereof. The term “functional portion” of a CDR can mean a sequence within the CDR which shows specific affinity for the target antigen. The functional portion of a CDR may comprise a ligand which specifically binds to the α2δ-1 peptide.

The term “CDR” can mean a hypervariable region in the heavy and light variable chains. There may be one, two, three or more CDRs in each of the heavy and light chains of the antibody. Normally, there are at least three CDRs on each chain which, when configured together, form the antigen-binding site, i.e. the three-dimensional combining site with which the antigen binds or specifically reacts. It has however been postulated that there may be four CDRs in the heavy chains of some antibodies.

The definition of CDR also includes overlapping or subsets of amino acid residues when compared against each other. The exact residue numbers which encompass a particular CDR or a functional portion thereof will vary depending on the sequence and size of the CDR. Those skilled in the art can routinely determine which residues comprise a particular CDR given the variable region amino acid sequence of the antibody.

The term “functional fragment” of an antibody can mean a portion of the antibody which retains a functional activity. A functional activity can be, for example antigen binding activity or specificity. A functional activity can also be, for example, an effector function provided by an antibody constant region. The term “functional fragment” is also intended to include, for example, fragments produced by protease digestion or reduction of a human monoclonal antibody and by recombinant DNA methods known to those skilled in the art. Human monoclonal antibody functional fragments include, for example individual heavy or light chains and fragments thereof, such as VL, VH and Fd; monovalent fragments, such as Fv, Fab, and Fab′; bivalent fragments such as F(ab′)₂; single chain Fv (scFv); and Fc fragments.

The term “VL fragment” can mean a fragment of the light chain of a human monoclonal antibody which includes all or part of the light chain variable region, including the CDRs. A VL fragment can further include light chain constant region sequences.

The term “VH fragment” can means a fragment of the heavy chain of a human monoclonal antibody which includes all or part of the heavy chain variable region, including the CDRs.

The term “Fd fragment” can mean the heavy chain variable region coupled to the first heavy chain constant region, i.e. VH and CH-1. The “Fd fragment” does not include the light chain, or the second and third constant regions of the heavy chain.

The term “Fv fragment” can mean a monovalent antigen-binding fragment of a human monoclonal antibody, including all or part of the variable regions of the heavy and light chains, and absent of the constant regions of the heavy and light chains. The variable regions of the heavy and light chains include, for example, the CDRs. For example, an Fv fragment includes all or part of the amino terminal variable region of about no amino acids of both the heavy and light chains.

The term “Fab fragment” can mean a monovalent antigen-binding fragment of a human monoclonal antibody that is larger than an Fv fragment. For example, a Fab fragment includes the variable regions, and all or part of the first constant domain of the heavy and light chains. Thus, a Fab fragment additionally includes, for example, amino acid residues from about 110 to about 220 of the heavy and light chains.

The term “Fab′ fragment” can mean a monovalent antigen-binding fragment of a human monoclonal antibody that is larger than a Fab fragment. For example, a Fab′ fragment includes all of the light chain, all of the variable region of the heavy chain, and all or part of the first and second constant domains of the heavy chain. For example, a Fab′ fragment can additionally include some or all of amino acid residues 220 to 330 of the heavy chain.

The term “F(ab′)₂ fragment” can mean a bivalent antigen-binding fragment of a human monoclonal antibody. An F(ab′)₂ fragment includes, for example, all or part of the variable regions of two heavy chains and two light chains, and can further include all or part of the first constant domains of two heavy chains and two light chains.

The term “single chain Fv (scFv)” can mean a fusion of the variable regions of the heavy (VH) and light chains (VL) connected with a short linker peptide.

The term “bispecific antibody (BsAb)” can mean a bispecific antibody comprising two scFv linked to each other by a shorter linked peptide.

According to a third aspect, there is provided an isolated nucleic acid encoding the peptide of the first aspect.

Preferably, the nucleic acid encodes a recombinant α2δ-1 peptide according to the first aspect, or comprises an amino acid sequence substantially as set out in SEQ ID No. 20.

According to a fourth aspect, there is provided a genetic construct comprising the nucleic acid according to the third aspect.

According to a fifth aspect, there is provided a recombinant vector comprising the genetic construct according to the fourth aspect.

According to a sixth aspect, there is provided a host cell comprising the genetic construct according to the fourth aspect, or the recombinant vector according to the fifth aspect.

According to a seventh aspect, there is provided a method of preparing an isolated α2δ-1 recombinant peptide, the method comprising (i) culturing at least one cell according to the sixth aspect; and (ii) isolating the peptide from the cell to create an isolated α2δ-1 recombinant peptide.

According to an eighth aspect, there is provided a membrane, micelle or liposome comprising the α2δ-1 peptide according to the first aspect, or the α2δ-1 peptide obtained or obtainable by the method according to the seventh aspect.

Preferably, the membrane, micelle, liposome or α2δ-1 peptide is recombinant. The membrane may be a plasma membrane or an organelle membrane.

According to a ninth aspect, there is provided a binding assay technology or identifying an agent that binds to the α2δ-1 protein of a voltage-gated calcium channel, the assay technology comprising the peptide according to the first aspect, or a peptide which comprises an amino acid sequence substantially as set out in SEQ ID No. 20.

The assay technology may comprise a positive control that binds to the isolated peptide. The positive control may be a drug, a peptide or an agent that binds to the isolated peptide of the kit. The positive control may be NVA1309, which is referred to herein as Formula I.

[(1R,5S,6S)-3-Ethyl-6-(1H-tetrazol-5-ylmethyl)-6-bicyclo[3.2.0]hept-3-enyl]methanamine Formula I

The kit may comprise a negative control that does not bind to the isolated peptide. The negative control may be a drug, a peptide or an agent that does not bind to the isolated peptide of the kit.

In a preferred embodiment, the kit may comprise a positive control and a negative control. The kit may also comprise further controls, as necessary. The kit may also comprise instructions for use.

The kit may comprise a peptide of the first aspect or a peptide referred to in the method of the eleventh aspect bound to a substrate. The peptide may be bound directly or indirectly by the substrate.

According to a tenth aspect, there is provided a genetically modified, non-human mammal comprising a mutation in the region of the wild-type α2δ-1 protein that corresponds to the sequence of SEQ ID No. 20.

The mutation may be one or more point mutations. Point mutations may be a substitution, an insertion, a deletion or a frameshift mutation.

The mutation may be a substitution or deletion of one or both of the lysines in the region of the α2δ-1 protein that corresponds to the sequence of SEQ ID No. 20. Preferably, the mutation is a deletion or substitution of the second lysine. The substitution may be a conservative substitution or a substitution for a similar amino acid.

The non-human mammal may be a carnivore (a cat or dog), a rodent (mouse or rat), a rabbit, a hare, a monkey or an ape.

According to a eleventh aspect, there is provided a method of identifying an agent that binds to the α2δ-1 protein of a voltage-gated calcium channel, the method comprising detecting for binding between the agent and the peptide according to the first aspect, or a peptide comprising an amino acid sequence substantially as set out in SEQ ID No. 20.

The method according to the eleventh aspect may be used to identify gabapentinoids and non-gabapentinoids that bind to the isolated peptide. Known methods of identifying novel gabapentinoids that bind to the α2δ-1 protein may involve detecting binding between a test agent (i.e. an agent suspected of being capable of binding to the isolated α2δ-1 peptide) and a membrane bound 3D-protein comprising the RRR motif, which is located upstream of the α2δ-1 peptide. However, the peptide according to the first aspect or referred to in the method of the eleventh aspect does not require a tertiary/three-dimensional (3D) structure in order to bind to gabapentinoids, such as NVA1309. This is advantageous because it simplifies the production and expression of the site (i.e. peptide/protein) to which the test agent may bind.

Furthermore, as a consequence of the peptides linear structure, it may be used in assays/techniques, such as Absorbance, Fluorescence intensity, Luminescence, Surface Plasmon Resonance (SPR), reverse Surface Plasmon Resonance (rSPR), Mass Spectrometry, Matrix-assisted Laser Desorption/Ionization Mass Spectrometry/Time of Flight (MALDI/TOF), Fluorescence Polarization, Fluorescence resonance energy transfer (FRET), Time resolved Fluorescence (TRF), Homogeneous Time Resolved Fluorescence (HTREF/TR-FRET), Alpha Screen Technology, Fluorescence lifetime, fragment complementation or FLIPR (for calcium readout) which may be incompatible with the tertiary/3D structure of the full-length α2δ-1 protein. Alternative assays/techniques that may be used to detect binding between the peptide of the first aspect (or referred to in the method of the eleventh aspect) may be ELISA, Radioligand binding assays and Immunoprecipitation.

Thus, the method according to the eleventh aspect may comprise using Absorbance, Fluorescence intensity, Luminescence, Surface Plasmon Resonance (SPR), reverse Surface Plasmon Resonance (rSPR), Mass Spectrometry, Matrix-assisted Laser Desorption/Ionization Mass Spectrometry/Time of Flight (MALDI/TOF), Fluorescence Polarization, Fluorescence resonance energy transfer (FRET), Time resolved Fluorescence (TRF), Homogeneous Time Resolved Fluorescence (HTREF/TR-FRET), Alpha Screen Technology, Fluorescence lifetime, fragment complementation, FLIPR, ELISA, Radioligand binding assays or Immunoprecipitation.

Preferably, prior to detecting for binding between the test agent and the peptide, the method according to the eleventh aspect also comprises the step of contacting the test agent and the peptide according to the first aspect, or a peptide comprising an amino acid sequence substantially as set out in SEQ ID No. 20.

The agent may be a gabapentinoid, a non-gabapentinoid, a peptide, a small molecule or an antibody.

A small molecule is an organic compound with a molecular weight equal to or less than 900 Daltons.

A gabapentinoid is an agent that may modulate (i.e. inhibit or enhance) the activity of a voltage-gated calcium channel that comprises an α2δ-1 subunit. Preferably, a gabapentinoid is an agent that modulates the activity of a voltage-gated calcium channel that comprises an α2δ-1 subunit. The activity of a voltage-gated calcium channel corresponds to a current generated in response to the passage of calcium ions through the pore-forming, α1 subunit of a voltage-gated channel. It will be appreciated that this activity can be measured using a variety of techniques known in the art, including patch clamping, voltage clamping and other electrophysiological techniques. Therefore, a gabapentinoid may be an agent that modulates the activity or the current of a voltage-gated calcium channel comprising an α2δ-1 subunit. Preferably, a gabapentinoid is a derivative of γ-aminobutyric acid (GABA), which comprises a GABA ring as defined by Formula II as follows:

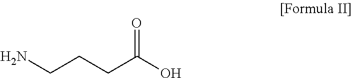

According to an twelfth aspect, there is provided an agent identified by the method according to the eleventh aspect, for use in therapy or as a medicament, or in diagnosis.

According to a thirteenth aspect, there is provided an agent identified by the method according to the eleventh aspect for use in the treatment of a medical condition in which the α2δ-1 subunit is a therapeutic target, the medical condition being selected from: pain, neuropathic pain, peripheral nervous system pain, central nervous system pain, hyperalgesia, tactile allodynia, fibromyalgia, restless legs syndrome, epilepsy, generalised anxiety disorder, migraine, social phobia, panic disorder, mania, bipolar disorder, and alcohol withdrawal, cancer, urinary tract infections, obstructive pulmonary disease, sexual dysfunction, Kawasaki disease, cardiovascular disorders, (such as angina, heart attacks, heart failure) and respiratory disorders (such as asthma and Chronic Obstructive Pulmonary Disease).

In a fourteenth aspect, there is provided a method of treating, preventing or ameliorating a condition in which the α2δ-1 subunit is a therapeutic target, the method comprising administering, to a subject in need of such treatment, a therapeutically effective amount of an agent identified by the method according to the eleventh aspect.

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A condition in which the α2δ-1 subunit is a therapeutic target may be a condition selected from: pain, neuropathic pain, peripheral nervous system pain, central nervous system pain, hyperalgesia, tactile allodynia, fibromyalgia, restless legs syndrome, epilepsy, generalised anxiety disorder, migraine, social phobia, panic disorder, mania, bipolar disorder, and alcohol withdrawal, cancer, urinary tract infections, obstructive pulmonary disease, sexual dysfunction, Kawasaki disease, cardiovascular disorders, (such as angina, heart attacks, heart failure) and respiratory disorders (such as asthma and Chronic Obstructive Pulmonary Disease).

According to an fifteenth aspect, there is provided a pharmaceutical composition of an agent, the composition comprising an agent identified by the method according to the eleventh aspect, and a pharmaceutically acceptable vehicle.

According to a sixteenth aspect, there is provided a use of a peptide comprising an isolated α2δ-1 peptide to identify an agent that binds thereto, wherein the peptide is the peptide according to the first aspect, or a peptide comprising an amino acid sequence substantially as set out in SEQ ID No. 20

The agent that binds thereto may be a gabapentinoid.

According to a seventeenth aspect, there is provided a use of an isolated α2δ-1 peptide to identify an agent that can be used to treat a medical condition in which the α2δ-1 subunit is a therapeutic target, wherein the peptide is the peptide according to the first aspect or comprises an amino acid sequence substantially as set out in SEQ ID No. 20.

The medical condition may be selected from: pain, neuropathic pain, peripheral nervous system pain, central nervous system pain, hyperalgesia, tactile allodynia, fibromyalgia, restless legs syndrome, epilepsy, generalised anxiety disorder, migraine, social phobia, panic disorder, mania, bipolar disorder, and alcohol withdrawal, cancer, urinary tract infections, obstructive pulmonary disease, sexual dysfunction, Kawasaki disease, cardiovascular disorders, (such as angina, heart attacks, heart failure) and respiratory disorders (such as asthma and Chronic Obstructive Pulmonary Disease).

It will be appreciated that agents, gabapentinoids, non-gabapentinoids and compositions according to the invention may be used in a medicament which may be used in a monotherapy, for treating, preventing or delaying the onset of any of the conditions mentioned herein. Alternatively, such agents, gabapentinoids, non-gabapentinoids and compositions according to the invention may be used as an adjunct to, or in combination with, known therapies for treating, preventing or delaying the onset of any of the conditions mentioned herein.

The agents, gabapentinoids and non-gabapentinoids according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising agents, gabapentinoids and non-gabapentinoids according to the invention may be used in a number of ways. For instance, oral administration may be required, in which case the agents may be contained within a composition that may, for example, be ingested orally in the form of a tablet, capsule or liquid. Compositions comprising gabapentinoids of the invention may be administered by inhalation (e.g. intranasally). Compositions may also be formulated for topical use. For instance, creams or ointments may be applied to the skin.

The agents, gabapentinoids, non-gabapentinoids and compositions according to the invention may also be incorporated within a slow- or delayed-release device. Such devices may, for example, be inserted on or under the skin, and the medicament may be released over weeks or even months. The device may be located at least adjacent to the treatment site. Such devices may be particularly advantageous when long-term treatment with agents used according to the invention is required and which would normally require frequent administration (e.g. at least daily injection).

In a preferred embodiment, agents, gabapentinoids, non-gabapentinoids and compositions according to the invention may be administered to a subject by injection into the blood stream or directly into a site requiring treatment. For example, the medicament may be injected at least adjacent to or within a tumour or a neuron. Injections may be intravenous (bolus or infusion) or subcutaneous (bolus or infusion), or intradermal (bolus or infusion).

It will be appreciated that the amount of the agent, the gabapentinoid, the non-gabapentinoid and the composition that is required is determined by its biological activity and bioavailability, which in turn depends on the mode of administration, the physicochemical properties of the modulator and whether it is being used as a monotherapy or in a combined therapy. The frequency of administration will also be influenced by the half-life of the agent or antibody within the subject being treated. Optimal dosages to be administered may be determined by those skilled in the art, and will vary with the particular agent in use, the strength of the pharmaceutical composition, the mode of administration, and the advancement of any of the conditions mentioned herein. Additional factors depending on the particular subject being treated will result in a need to adjust dosages, including subject age, weight, gender, diet, and time of administration.

Generally, a daily dose of between 0.01 μg/kg of body weight and 500 mg/kg of body weight of the agent according to the invention may be used for treating, ameliorating, or preventing any of the conditions mentioned herein, depending upon which agent is used. More preferably, the daily dose is between 0.01 mg/kg of body weight and 400 mg/kg of body weight, more preferably between 0.1 mg/kg and 200 mg/kg body weight, and most preferably between approximately 1 mg/kg and 100 mg/kg body weight.

The agent, gabapentinoid, non-gabapentinoids or composition may be administered before, during or after onset of the conditions mentioned herein. Daily doses may be given as a single administration (e.g. a single daily injection). Alternatively, the agent, the gabapentinoid, the non-gabapentinoid and the composition may require administration twice or more times during a day. As an example, agents may be administered as two (or more depending upon the severity of the disease being treated) daily doses of between 25 mg and 7000 mg (i.e. assuming a body weight of 70 kg). A subject receiving treatment may take a first dose upon waking and then a second dose in the evening (if on a two dose regime) or at 3- or 4-hourly intervals thereafter. Alternatively, a slow release device may be used to provide optimal doses of agents according to the invention to a patient without the need to administer repeated doses.

Known procedures, such as those conventionally employed by the pharmaceutical industry (e.g. in vivo experimentation, clinical trials, etc.), may be used to form specific formulations comprising the agents according to the invention and precise therapeutic regimes (such as daily doses of the agents and the frequency of administration).

A “therapeutically effective amount” of agent is any amount which, when administered to a subject, is the amount of the agent, the gabapentinoid, the non-gabapentinoid and the compositions that is needed to treat any of the conditions mentioned herein, or produce the desired effect, such as inhibiting neuropathic pain.

For example, the therapeutically effective amount of agent used may be from about 0.01 mg to about 800 mg, and preferably from about 0.01 mg to about 500 mg. It is preferred that the amount of agent is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g. the peptide or antibody) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidone, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The active agent according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and Arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilised by, for example, intramuscular, intrathecal, epidural, intraperitoneal, intravenous and particularly subcutaneous injection. The agent, composition or antibody may be prepared as a sterile solid composition that may be dissolved or suspended at the time of administration using sterile water, saline, or other appropriate sterile injectable medium.

The agents and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The agent, the gabapentinoid, the non-gabapentinoid and the composition according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the nucleic acids or polypeptides described herein.

Amino acid/polynucleotide/polypeptide sequences with a sequence identity which is greater than 50%, more preferably greater than 65%, 70%, 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90%, 92%, 95%, 97%, 98%, and most preferably at least 99% identity with any of the sequences referred to herein. The amino acid/polynucleotide/polypeptide sequence may have 100% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on: (i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula: Sequence Identity=(N/T)*100.

Alternative methods for identifying similar sequences will be known to those skilled in the art. For example, a substantially similar nucleotide sequence will be encoded by a sequence which hybridizes to any sequences referred to herein or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the polypeptide sequences described herein.

Due to the degeneracy of the genetic code, it is clear that any nucleic acid sequence described herein could be varied or changed without substantially affecting the sequence of the protein encoded thereby, to provide a variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

### Amino Acid Substitutions:

Amino acid substitutions may be made to a peptide sequence, for example up to 1, 2, 3, 4, 5, 10, 20 or 30 substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality, or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | |
|---|---|
| Ala | aliphatic, hydrophobic, neutral |
| Cys | polar, hydrophobic, neutral |
| Asp | polar, hydrophilic, charged (−) |
| Glu | polar, hydrophilic, charged (−) |
| Phe | aromatic, hydrophobic, neutral |
| Gly | aliphatic, neutral |
| His | aromatic, polar, hydrophilic, |
| | charged (+) |
| Ile | aliphatic, hydrophobic, neutral |
| Lys | polar, hydrophilic, charged(+) |
| Leu | aliphatic, hydrophobic, neutral |
| Met | hydrophobic, neutral |
| Asn | polar, hydrophilic, neutral |
| Pro | hydrophobic, neutral |
| Gln | polar, hydrophilic, neutral |
| Arg | polar, hydrophilic, charged (+) |
| Ser | polar, hydrophilic, neutral |
| Thr | polar, hydrophilic, neutral |
| Val | aliphatic, hydrophobic, neutral |
| Trp | aromatic, hydrophobic, neutral |
| Tyr | aromatic, polar, hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

Conservative substitutions are those in which at least one residue in the amino acid sequence has been removed and a different residue inserted in its place. Such substitutions generally are made in accordance with the Table 3 when it is desired to maintain the activity of the protein. Table 2 shows amino acids which can be substituted for an amino acid in a protein and which are typically regarded as conservative substitutions.

TABLE 3

| | Conservative |
|---|---|
| Residue | Substitutions |
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Similar substitutions are those in which at least one residue in the amino acid sequence has been removed and a different residue inserted in its place. Such substitutions generally are made in accordance with the Table 4 when it is desired to maintain the activity of the protein. Table 4 shows amino acids which can be substituted for an amino acid in a protein and which are typically regarded as structural and functional substitutions. For example, a residue in column 1 of Table 4 may be substituted with a residue in column 2; in addition, a residue in column 2 of Table 4 may be substituted with the residue of column 1.

TABLE 4

| Residue | Similar Substitutions |
|---|---|
| Ala | Ser; Thr; Gly; Val; Leu; Ile |
| Arg | Lys; His; Gly |
| Asn | Gln; His; Gly; Ser; Thr |
| Asp | Glu, Ser; Thr |
| Gln | Asn; Ala |
| Cyc | Ser; Gly |
| Glu | Asp |
| Gly | Pro; Arg |
| His | Asn; Gln; Tyr; Phe; Lys; Arg |
| Ile | Ala; Leu; Val; Gly; Met |
| Leu | Ala; Ile; Val; Gly; Met |
| Lys | Arg; His; Gln; Gly; Pro |
| Met | Leu; Ile; Phe |
| Phe | Met; Leu; Tyr; Trp; His; Val; Ala |
| Ser | Thr; Gly; Asp; Ala; Val; Ile; His |
| Thr | Ser; Val; Ala; Gly |
| Trp | Tyr; Phe; His |
| Tyr | Trp; Phe; His |
| Val | Ala; Ile; Leu; Gly; Thr; Ser; Glu |

Substitutions that are less conservative than those in Table 2 can be selected by picking residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, for example, seryl or threonyl, is substituted for (or by) a hydrophobic residue, for example, leucyl, isoleucyl, phenylalanyl, valyl, or alanyl; (b) a cysteine or proline is substituted for (or by) any other residue; (c) a residue having an electropositive side chain, for example, lysyl, arginyl, or histidyl, is substituted for (or by) an electronegative residue, for example, glutamyl or aspartyl; or (d) a residue having a bulky side chain, for example, phenylalanine, is substituted for (or by) one not having a side chain, for example, glycine.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:

FIG. 1 is a schematic showing the topological domains/features of the human α2δ-1 subunit, which is encoded by CACNA2D1.

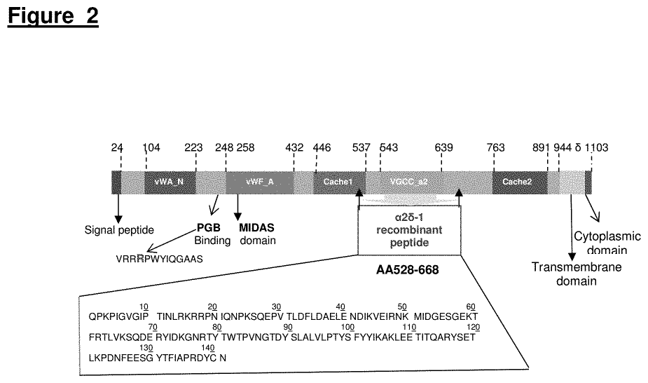

FIG. 2 is a schematic showing domain organisation of the human α2δ-1 subunit. It specifically shows the topology of CACNA2D1 domains and the α2δ-1 recombinant peptide sequence within the VGCC_α2 domain.

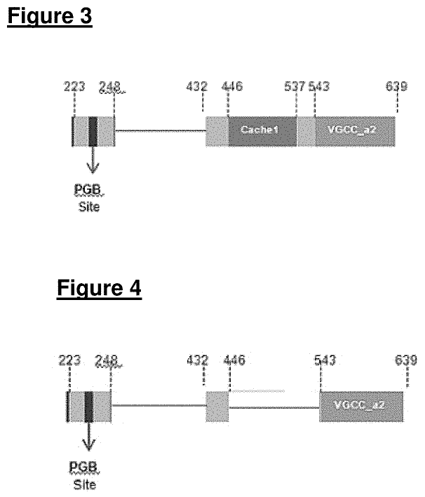

FIG. 3 is a schematic representation of construct 1 (SEQ ID No. 2). It is a truncated α2δ-1 fragment containing the gabapentin/pregabalin binding site motif but lacking the VWA_N, VWF_A and carboxy-terminal regions downstream of VGGC-α2 domain.

FIG. 4 is a schematic representation of construct 2 (SEQ ID No. 3). It is a truncated α2δ-1 fragment containing the gabapentin/pregabalin binding site motif but lacking the VWA_N, VWF_A and carboxy-terminal regions downstream of VGGC-α2 domain and also lacking the Cache 1 domain.

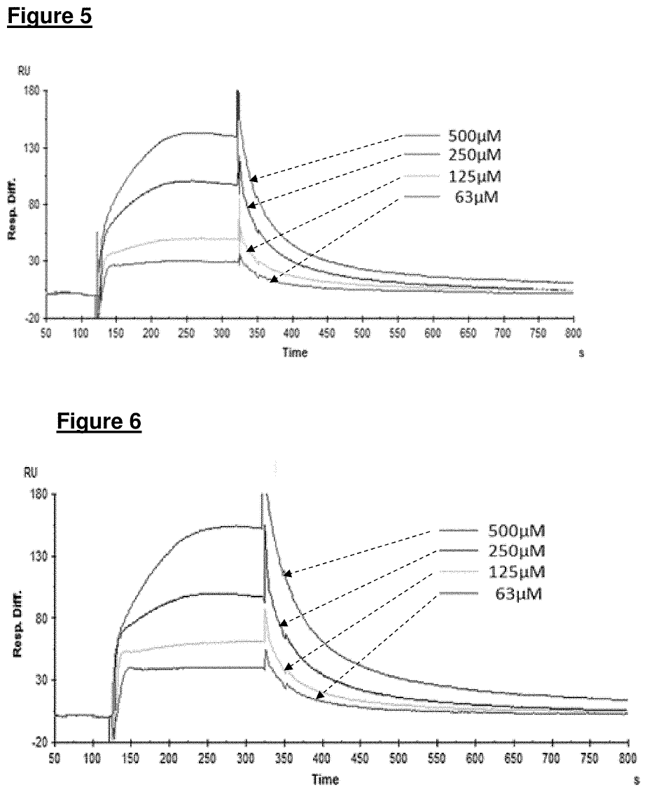

FIG. 5 shows SPR sensorgrams for binding of NVA1309 to immobilized α2δ-1 construct 1.

FIG. 6 shows SPR sensorgrams for binding of NVA1309 to immobilized α2δ-1 construct 2.

FIG. 7 shows SPR sensorgrams for binding of pregabalin to immobilized α2δ-1 constructs 1 and 2.

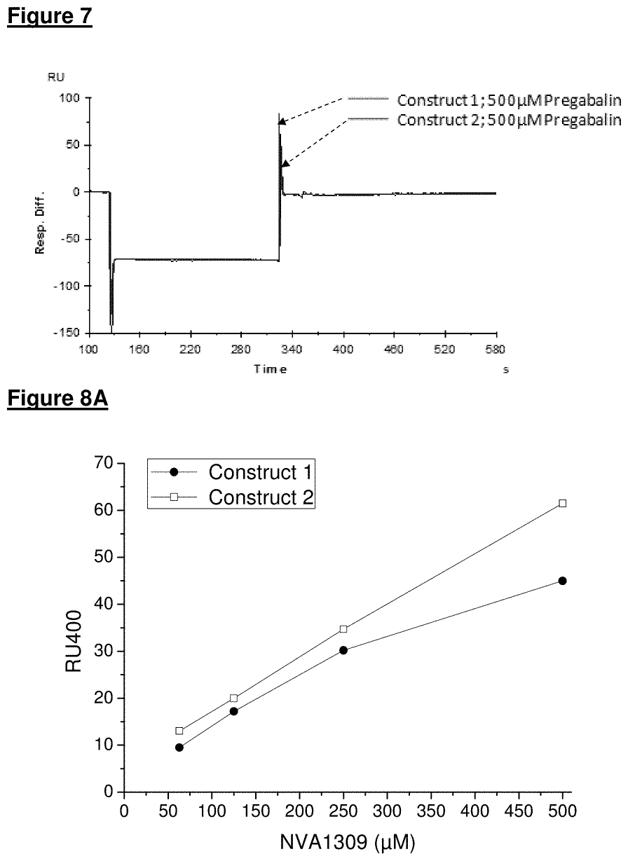

FIGS. 8A and 8B show specific SPR binding of compound NVA1309 and pregabalin to truncated α2δ-1 Constructs 1 and 2.

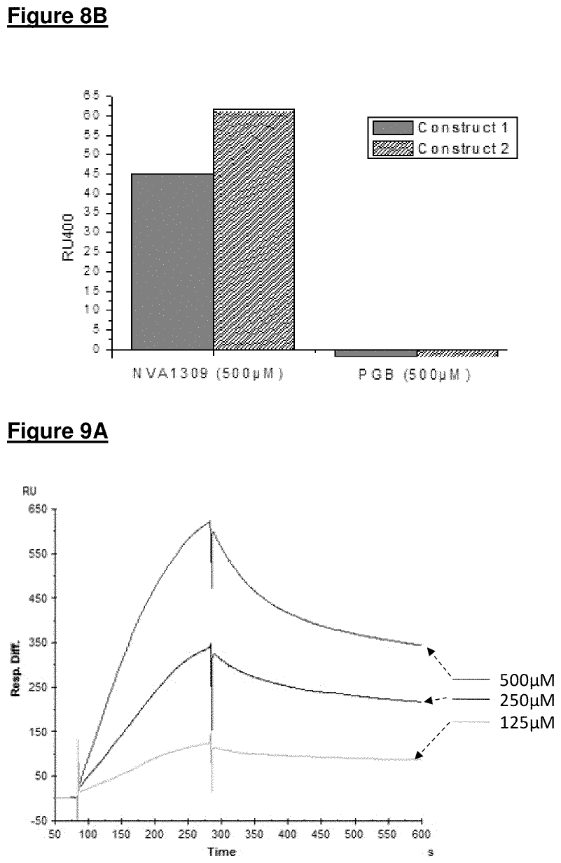

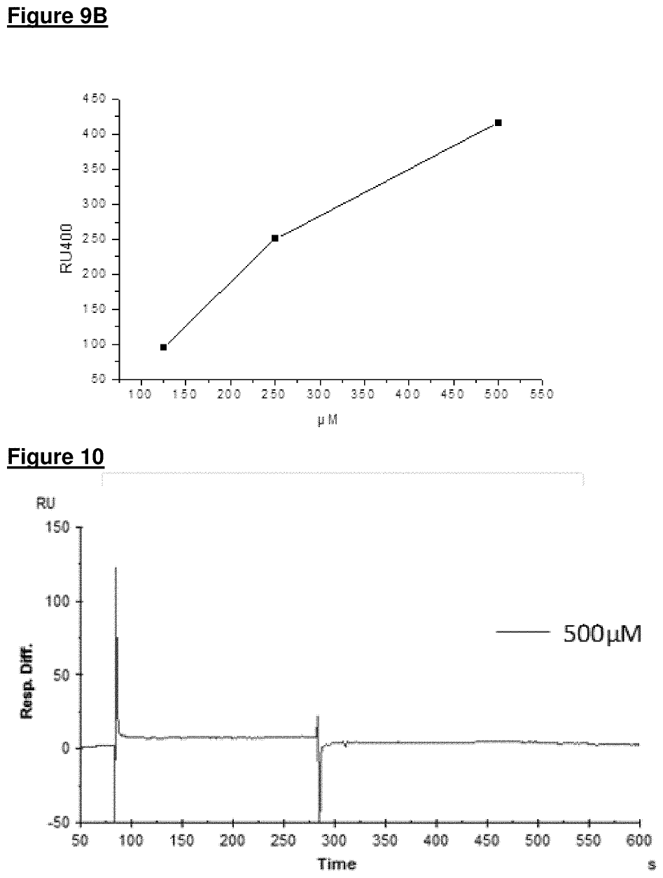

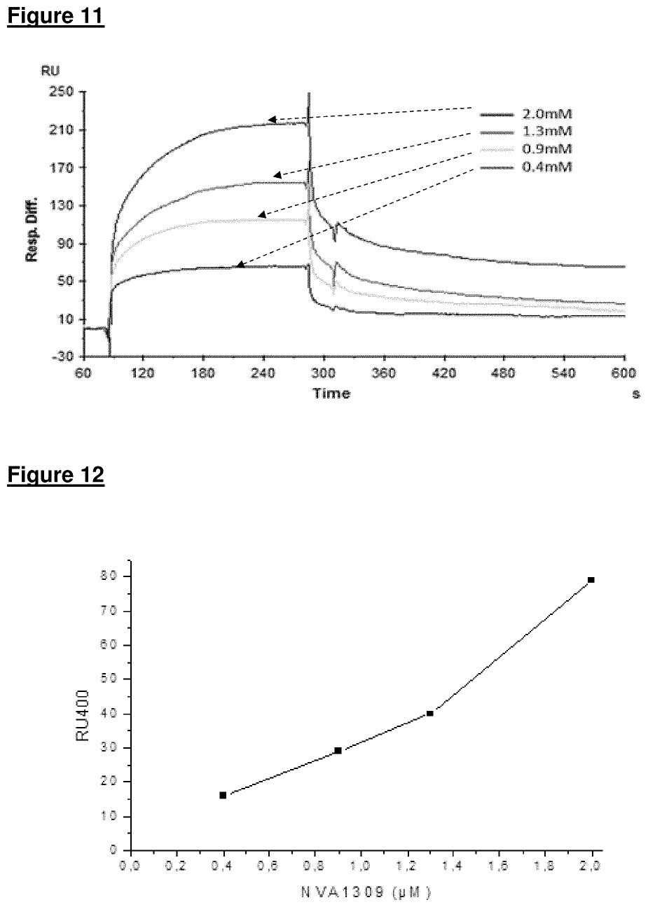

FIG. 9A shows blank-subtracted sensorgram overlay. SPR sensorgrams showing binding of NVA1309 to immobilized peptide 1 (containing the gabapentin/pregabalin binding site).

FIG. 9B shows RU400 values of FIG. 9A.

FIG. 10 shows SPR sensorgrams for binding of pregabalin to immobilized peptide 1 (P1).

FIG. 11 shows SPR sensorgrams for binding of NVA1309 to immobilized an α2δ-1 recombinant peptide containing the VGGC_α2 domain.

FIG. 12 shows specific SPR binding of compound NVA1309 to immobilized an α2δ-1 recombinant peptide containing the VGGC_α2 domain.

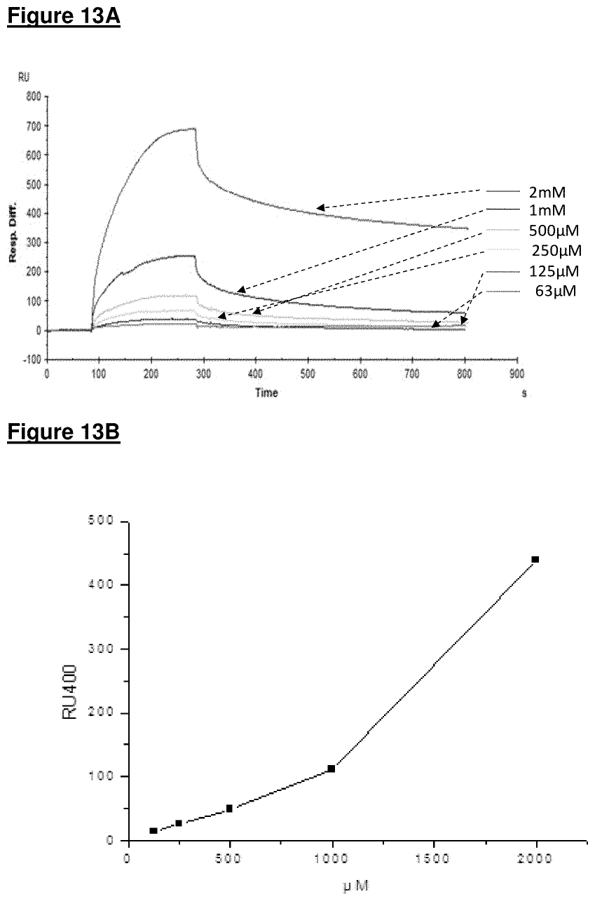

FIG. 13A shows SPR sensorgrams for binding of NVA1309 to immobilized peptide 3 (carboxy terminal VGGC_α2 region (I)). Blank-subtracted sensorgram overlay. FIG. 13B shows RU400 values of FIG. 13A.

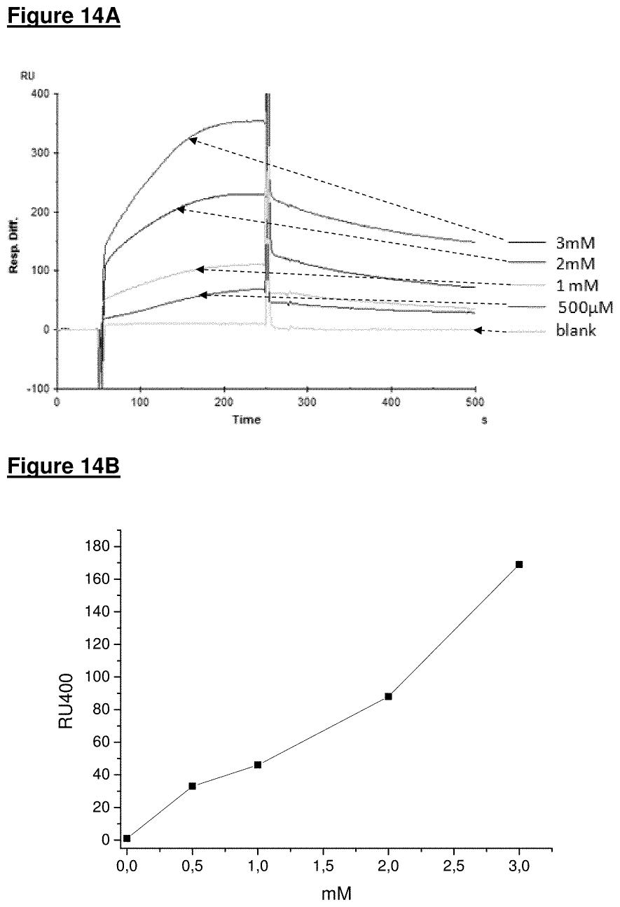

FIG. 14A shows SPR sensorgrams for binding of NVA1309 to immobilized peptide 4. Blank—subtracted sensorgram overlay. FIG. 14B shows RU400 values of FIG. 14A.

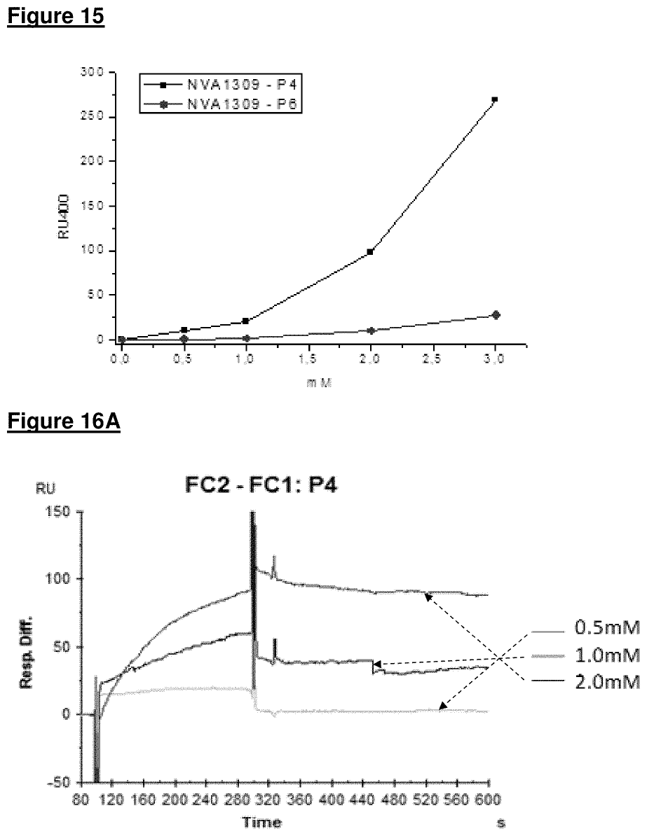

FIG. 15 is an RU400 plot for binding of NVA1309 to immobilized peptides P4 and P6.

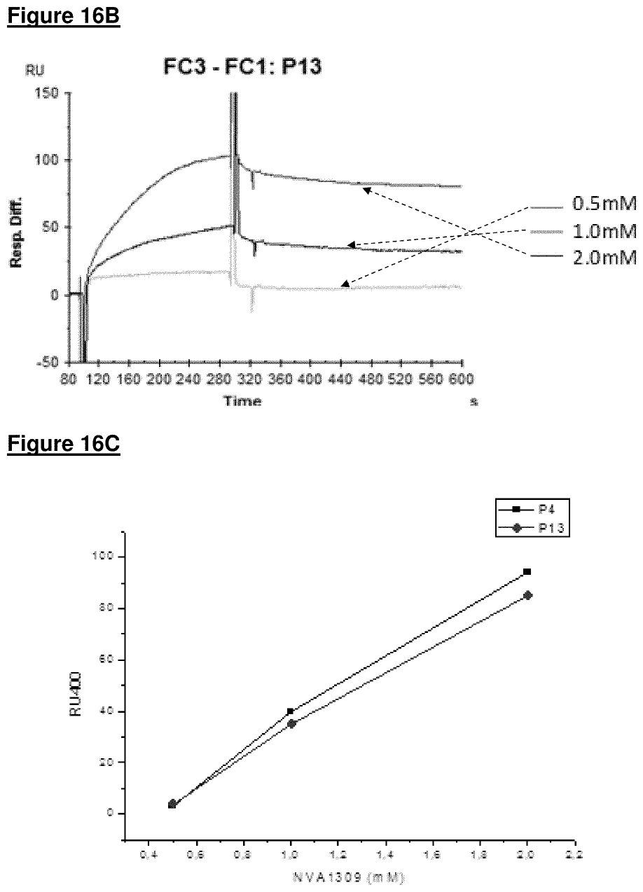

FIG. 16A shows binding of NVA1309 to peptide P4: Sensorgram overlay. FIG. 16B shows binding of NVA1309 to peptide P13: Sensorgram overlay. FIG. 16C shows binding of NVA1309 to peptides P4 and P13: RU400 plot.

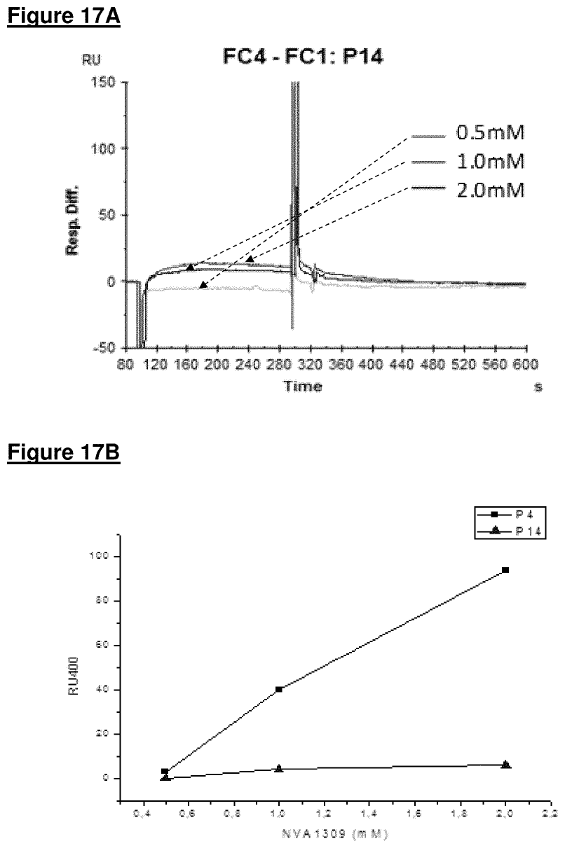

FIG. 17A shows binding of NVA1309 to peptide P14: Sensorgram overlay. FIG. 17B shows binding of NVA1309 to peptides P4 and P14: RU400 plot.

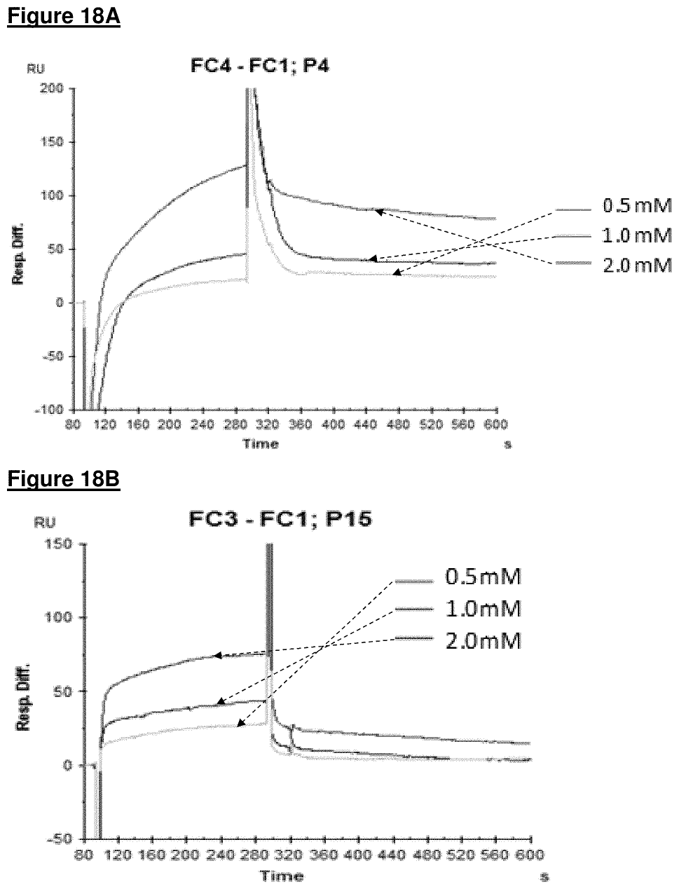

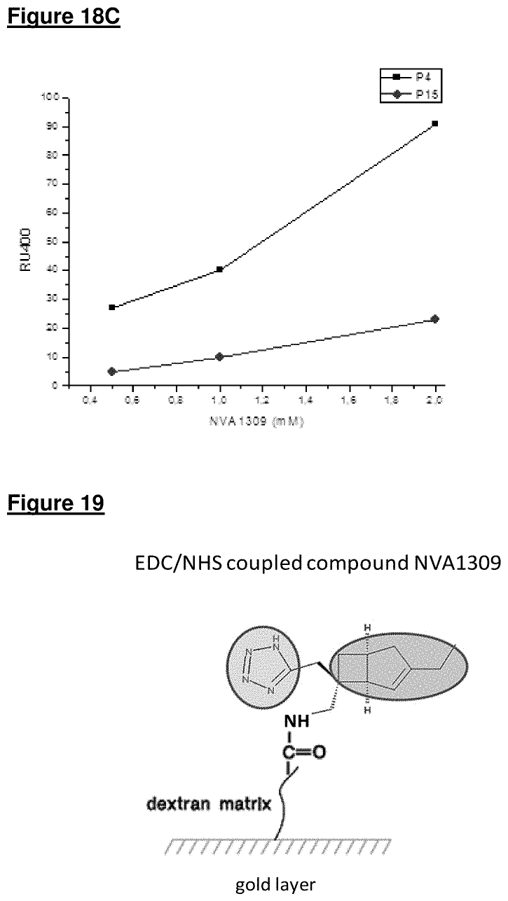

FIG. 18A shows binding of NVA1309 to peptide P4: Sensorgram overlay. FIG. 18B shows binding of NVA1309 to peptide P15: Sensorgram overlay. FIG. 18C shows binding of NVA1309 to peptides P4 and P15: RU400 plot.

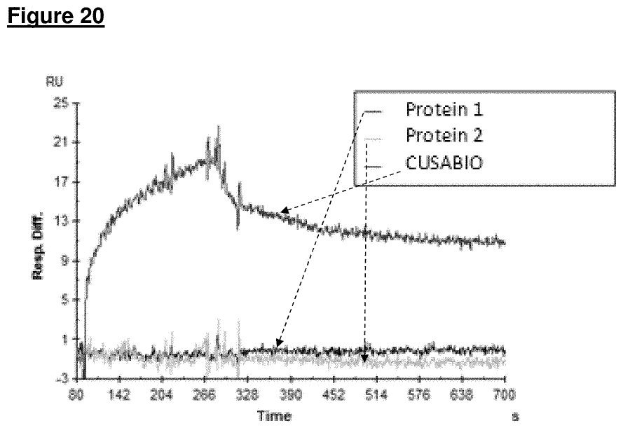

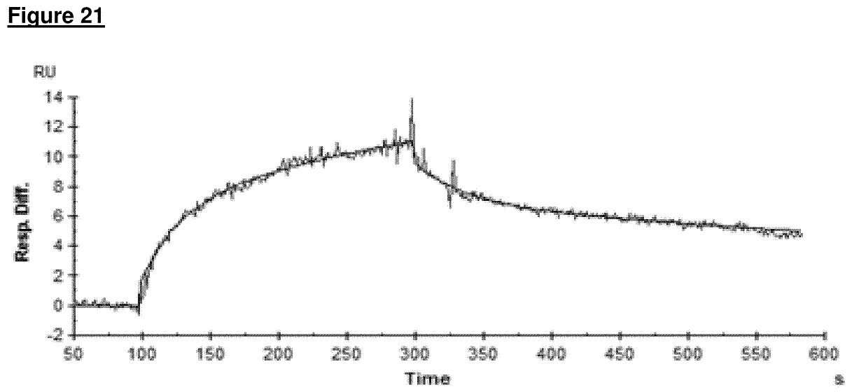

FIG. 19 shows covalent immobilization of compound NVA1309 on the Biacore CM5 sensor chip surface.

FIG. 20 shows specific SPR binding of “an α2δ-1 recombinant peptide comprising the VGGC_α2 domain to NVA1309, covalently coupled to the surface of a Biacore CM5 optical sensor chip.

FIG. 21 shows mathematical sensorgram fitting (Langmuir 1:1 binding model) of the α2δ-1 recombinant peptide/NVA1309 binding curve.

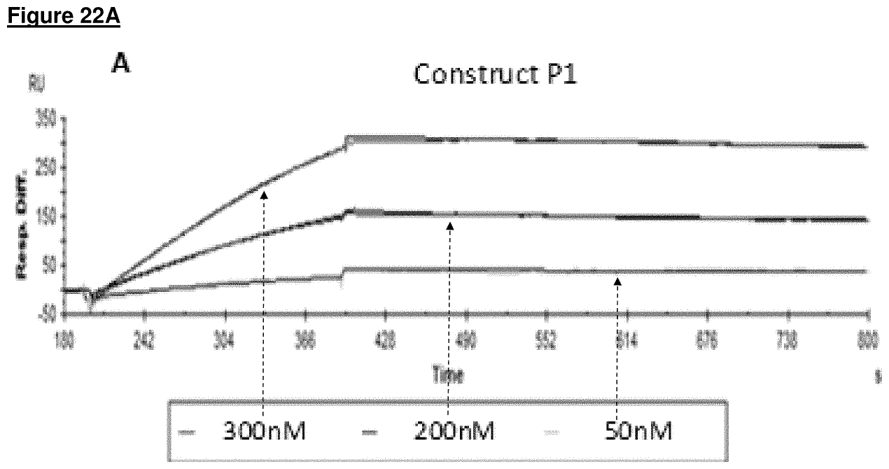

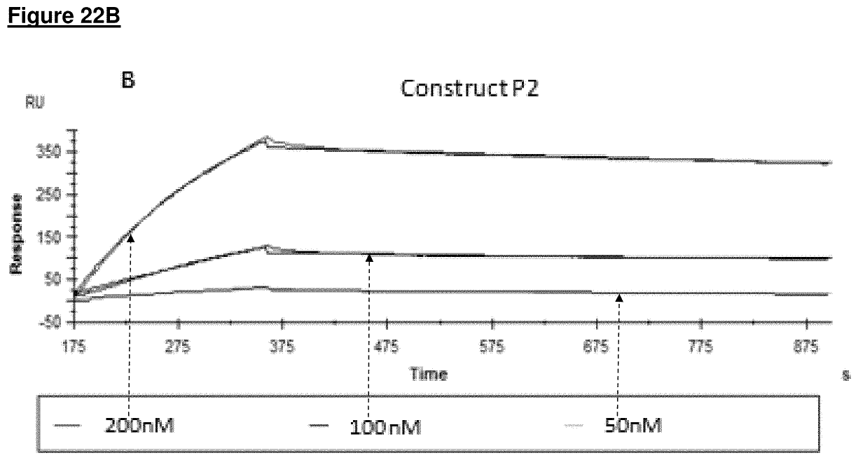

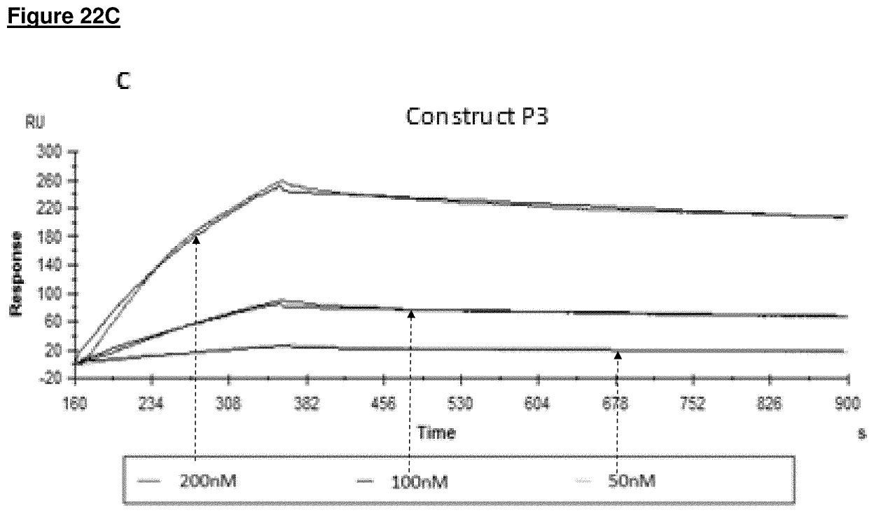

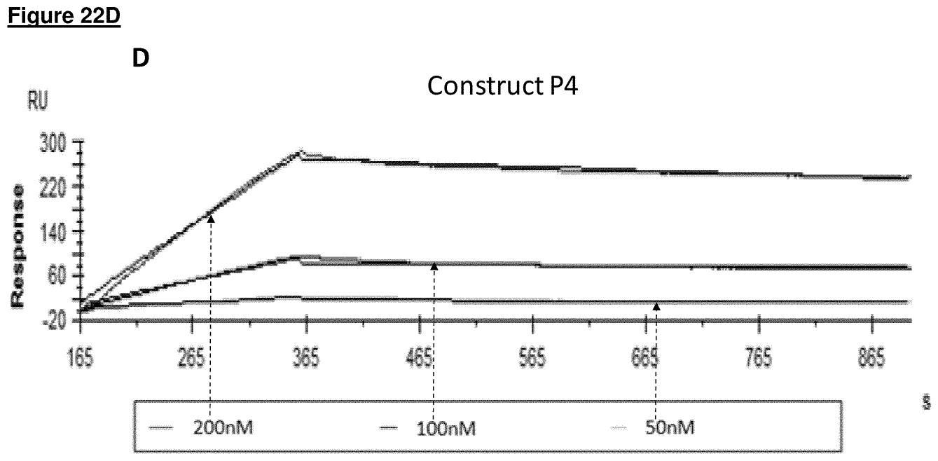

FIG. 22(A to D) shows SPR sensorgram fitting of α2δ-1 constructs (P1-P4) encoding human recombinant α2δ-1 proteins (PR1-4)—NVA1309 binding curves.

### EXAMPLES

The inventors decided to explore the significance of individual α2δ-1 domains in the binding profile of the gabapentinoid, NVA1309. Pregabalin and Gabapentin were used as comparative examples. Two truncated α2δ-1 proteins were generated by recombinant synthetic DNA cloning and transiently expressed as His-tagged GST fusion proteins in CHO cells. Also, a CACNA2D1 (human α2δ-1) recombinant peptide produced in yeast (CACNA2D1; CUSABIO, Cat. No. CSB-YP004407HU), lacking the predominant pregabalin binding region as established in the prior art [13] has also been used. FIG. 2 shows a schematic the topology of CACNA2D1 domains and the α2δ-1 recombinant peptide sequence comprising the VGCC_α2 domain.

Furthermore, the α2δ-1 recombinant peptide together with a set of synthetic peptides representing defined α2δ-1 regions and mutant derivatives thereof were used in target binding experiments. Using Surface Plasmon Resonance (SPR) technology for real time binding assessment, it was found that NVA1309 to bind to a recombinant full length α2δ-1 protein with high affinity but has also been unexpectedly shown to bind the α2δ-1 recombinant peptide comprising the VGCC_α2 domain but not the known binding site for gabapentinoids (R217). Compound NVA1309 was also specifically binding to a synthetic peptide comprising the pregabalin/gabapentin binding site described in prior art (R217) and also was found to bind independently to a site within the carboxy-terminal amino acid region of the α2δ-1VGCC_α2 domain, contained within the α2δ-1 recombinant peptide. This novel and unexpected finding postulates the existence of a novel and independent target binding site for gabapentinoid compounds on human CACNA2D1 which has not been identified in the prior art. The supporting data associated with these unexpected findings are discussed in detail in the following Examples.

### Surface Plasmon Resonance (SPR)

Binding of the gabapentinoid, NVA1309, to recombinant fragments and synthetic peptides comprising different regions of the α2δ-1 target molecule was investigated using Surface Plasmon Resonance (SPR; Biacore) technology.

Biomolecular interaction analysis by the SPR technology applies optical sensor chips and allows to study in real time binding events in between molecules. Only μg amounts of non-labelled proteins (e.g. antibodies/antigens, recombinant receptors, ligands) or low molecular compounds (e.g. drug candidates) are sufficient to assess target binding strength and to determine accurate binding kinetics (on/off rates, affinities).

### Example 1—Binding of Gabapentinoid NVA1309 and Pregabalin to Truncated Recombinant α2δ-1 Proteins, Construct 1 (SEQ ID No. 2) and Construct 2 (SEQ ID No. 3)

Binding experiments were carried out using Surface Plasmon Resonance (SPR) technology in a Biacore instrument. Recombinant construct 1 and construct 2 were immobilized on the surface of two different flow cells (FC2 and FC3, respectively) of a Biacore CM5 optical sensor chip by covalent amine coupling chemistry, using the Biacore amine coupling kit and following the Biacore amine coupling protocol. Human IgG was immobilized to the reference flow cell FC1 as intra-assay background binding control. Compound NVA1309 was injected as analyte at increasing concentrations and the binding reaction was monitored by generation of real time sensorgrams.

Two recombinant truncated α2δ-1 fragments (constructs 1 and 2; see FIGS. 3 and 4), containing the reported pregabalin binding site were found to bind NVA1309 but lacked binding to pregabalin. These two truncated α2δ-1 proteins were generated by recombinant synthetic DNA cloning and transiently expressed as his-tagged GST fusion proteins in CHO cells.

### Construct 1

Construct 1 (SEQ ID No. 2) lacks:
a) the amino terminal VWA_N domain, which is found at the N-terminus of proteins containing von Willebrand factor type A (VWA) and Cache domains. It has been found in vertebrates, Drosophila and C. Elegans but has not yet been identified in other eukaryotes. It is probably involved in the function of some voltage-dependent calcium channel subunits;
b) the complete Von Willebrand factor type A domain; and
c) all carboxy-terminal regions downstream of VGGC-α2 domain, which includes (i) the gabapentin/pregabalin binding site motif, (ii) the Cache 1 domain, an extracellular protein domain predicted to play a role in small-molecule recognition in a wide range of proteins, including α2δ-1 and various bacterial chemotaxis receptors, and (iii) the VGGC_α2 domain, a specific domain present in various neuronal voltage-dependent calcium channel (VGCC) subunits to the N-terminus of a Cache domain.

### Construct 2

Construct 2 (SEQ ID No. 3) is the same as construct 1 but also lacking the Cache 1 domain.

Sensorgram running conditions were:
Compound stock solution: 20 mM, HBS-P buffer
Compound working stock concentrations 0.66-3.62 mM
Running buffer: HBS-P
Flow rate: 30 μl/min

Results are presented as subtracted curves from the human IgG reference in FIG. 5 and FIG. 6.

Pregabalin was injected as analyte at a concentration of 500 μM, corresponding to the highest concentration applied for compound NVA1309 (see FIG. 7).

Subtractive binding sensorgrams clearly show lack of pregabalin binding to both the truncated α2δ-1 ligands although both truncated proteins contained an intact gabapentin/pregabalin binding motif (RRR) (see FIGS. 3 and 4). Similar data were obtained using gabapentin as analyte, with no binding observed to occur for both α2δ-1 Constructs 1 and 2.

Taking the relative SPR Response Units measured at 400 sec. after injection (RU400; early dissociation phase) as a reference report point, concentration-dependent binding of compound NVA1309 and lack of pregabalin binding to the truncated recombinant α2δ-1 constructs is clearly demonstrated. Specific SPR binding of NVA1309 and pregabalin to truncated α2δ-1 fragments is illustrated in FIGS. 8A & 8B.

### Example 2—Binding of Gabapentinoid Compound NVA1309 to a Synthetic Peptide Comprising the Gabapentin/Pregabalin Binding Site of α2δ-1

A synthetic peptide was generated comprising the reported gabapentin/pregabalin binding site upstream of the VWF_A domain of α2δ-1, carboxy-terminally fused to a flexible spacer consisting of three glycines and a terminal cysteine (Peptide 1, P1; SEQ ID No. 4):

| [SEQ ID No: 4] | |
|---|---|
| P1: | RTPNKIDLYDVRRRPWYIQGAGGGC |

This peptide was covalently coupled via the carboxy-terminal cysteine to the surface (FC2) of a Biacore CM5 optical sensor chip using the Biacore Thiol Coupling Kit, following the Biacore thiol coupling procedure. Thiol coupling allows for uniform surface presentation of the immobilized peptide molecules and provides freedom to adopt a steric conformation that is determined by the amino acid sequence of the peptide. Compound NVA1309 was injected as analyte at increasing concentrations and the binding reaction was monitored by generation of real time sensorgrams, presented as subtracted curves from the blank FC1 dextran reference surface.

Sensorgram running conditions were:
Compound stock solution: 50 mM, DMSO
Compound working stock dilution: 0.5-2 mM
Running buffer: HBS-P
Flow rate: 30 μl/min

Taking the relative SPR Response Units measured at 400 sec. after injection (RU400; early dissociation phase) as a reference report point, concentration-dependent binding of compound NVA1309 to the synthetic peptide comprising the reported gabapentin/pregabalin binding site is clearly demonstrated (see FIGS. 9A & 9B).

Pregabalin as analyte yielded no positive SPR signal for binding to the peptide P1 ligand and this has confirmed the data shown in Example 1 (see FIG. 10).

### Example 3—Binding of Gabapentinoid Compound NVA1309 to the α2δ-1 Recombinant Peptide (SEQ ID No. 5) Comprising the VGCC_α2 Domain but not the Known Gabapentin/Pregabalin Binding Site

The α2δ-1 recombinant peptide comprising the VGGC_α2 domain and containing a single cysteine near the carboxy terminus, was immobilized on the surface of a Biacore CM5 optical sensor chip using the Biacore Thiol Coupling Kit following the covalent thiol coupling chemistry. This immobilization process provides a sterically more uniform attachment of the ligand molecules to the sensor chip surface than the randomized amine coupling procedure. Bovine serum albumin (BSA) was immobilized to the reference flow cell FC1 as intra-assay background binding control. Compound NVA1309 was injected as analyte at increasing concentrations and the binding reaction was monitored by generation of real time sensorgrams, presented as subtracted curves from the BSA reference (see FIG. ii).

Sensorgram running conditions were:
Compound stock solution: 50 mM, Dimethyl sulfoxide (DMSO) DMSO
Compound working dilution: 0.5-2 mM
Running buffer: HBS-P
Flow rate: 30 μl/min

The relative SPR Response Units measured at 400 sec. after injection (RU400; early dissociation phase) as reference report points, a concentration-dependent binding of compound NVA1309 to the α2δ-1 recombinant peptide is clearly demonstrated (see FIGS. 11 and 12).

### Example 4—Specific Binding of Gabapentinoid Compound NVA1309 to a Synthetic Peptide Comprising the Carboxy Terminal Amino Acid Region of the VGGC_α2 Domain of α2δ-1

A synthetic peptide was constructed, representing the carboxy terminal of the VGGC_α2 domain of α2δ-1, carboxy-terminally fused to a flexible spacer consisting of three glycines and a terminal cysteine (Peptide 3; SEQ ID No. 6):

Peptide P₃, carboxy-terminal VGGC_α2 region
(I) + GGGC anchor
[SEQ ID No. 6]

| | |
|---|---|
| P3: | TYSFYIKAKLEETITQARYSETGGGC |

This peptide was covalently coupled via the carboxy-terminal cysteine to the surface (FC2) of a Biacore CM5 optical sensor chip using the Biacore Thiol Coupling Kit following the Biacore thiol coupling procedure. This strategy allows for uniform surface presentation of the immobilized peptide molecules and provides freedom to adopt a steric conformation determined by the amino acid sequence of the peptide. Compound NVA1309 was injected as analyte at increasing concentrations and the binding reaction was monitored by generation of real time sensorgrams, presented as subtracted curves from the blank FC1 dextran reference surface (see FIG. 13A).

Sensorgram running conditions were:
Compound stock solution: 50 mM, Dimethyl sulfoxide (DMSO) DMSO
Compound working dilution: 0.5-2 mM
Running buffer: HBS-P (Biacore)
Flow rate: 30 μl/min

Taking the relative SPR Response Units measured at 400 sec. after injection (RU400; early dissociation phase) as reference report points, concentration dependent binding of compound NVA1309 to peptide P3, homologous to the carboxy-terminal end of the α2δ-1 VGGC_α2 region (I) domain is clearly demonstrated (see FIG. 13B).

### Example 5—Mapping of the Gabapentinoid Compound NVA1309 Binding Site on the Carboxy Terminal Region of the VGGC_α2 Target Domain: Binding to Core Amino Acid Sequence IKAKLEETITQA

A synthetic peptide was constructed comprising the core amino acid sequence IKAKLEETITQAGGGC contained in the Peptide 3 (SEQ ID No. 6), carboxy-terminally fused to the flexible spacer consisting of three glycines and a terminal cysteine (Peptide 4; SEQ ID No. 7):

| [SEQ ID No. 7] | |
|---|---|
| P4: | IKAKLEETITQAGGGC |

This peptide was covalently coupled via the carboxy-terminal cysteine to the surface (FC2) of a Biacore CM5 optical sensor chip as described in Example 4 following the Biacore thiol coupling procedure. Compound NVA1309 was injected as analyte at increasing concentrations and the binding reaction was monitored by generation of real time sensorgrams, presented as subtracted curves from the blank FC1 dextran reference surface (FIGS. 14A & 14B).

Sensorgram running conditions were:
Compound stock solution: 50 mM, Dimethyl sulfoxide (DMSO)
Compound working dilution: 0.5-2 mM
Running buffer: HBS-P
Flow rate: 30 μl/min

An additional peptide was synthesized starting with the carboxy-terminal half of peptide P4 extended to the final downstream VGGC_α2 amino acids, carboxy-terminally fused to the flexible spacer consisting of three glycines and a terminal cysteine: (Peptide P6; SEQ ID No. 8):

| [SEQ ID No. 8] | |
|---|---|
| P6: | ETITQARYSETLKPGGGC |

This peptide was covalently coupled via the carboxy-terminal cysteine to the surface (FC3) of a new Biacore CM5 optical sensor chip using the Biacore thiol coupling procedure. Peptide P4 was immobilized on FC2 as a positive reference control, flow cell FC1 was left blank as negative background binding reference. Compound NVA1309 was injected as analyte at increasing concentrations and the binding reaction was monitored by generation of real time sensorgrams as described in the previous examples and presented as subtracted curves from the blank FC1 dextran reference surface.

The relative SPR R400 report point Response Units obtained from the blank subtracted sensorgrams for peptides P4 and P6 are shown in direct comparison in FIG. 15.

The results of the experiments disclosed in Example 5 clearly demonstrate that the amino acid sequence stretch IKAKLEETITQA at the carboxy-terminal end of the α2δ-1 VGGC_α2 domain defines the novel binding site for compound NVA1309. Deletion of the amino-terminal part of this sequence and further extension of the carboxy-terminal end of VGGC_α2 as represented in peptide P6 (SEQ ID No. 8) resulted in significant loss of NVA1309 binding capacity. Thus, the amino-terminal part of peptide P4 (SEQ ID No. 7), IKAKLE contributes to compound NVA1309 target binding.

### Example 6: Molecular Characterisation of the Novel VGGC_α2 Gabapentinoid Binding Site (Peptide P4 Scanning)

A series of three concentration SPR binding experiments were carried out with peptides derived from the peptide P4 sequence (SEQ ID No. 7). These peptides were designed in order to determine the significance of single peptide 4-contained (P4) amino acid residues in binding compound NVA1309. The following peptides were prepared:

### Peptide P8 (SEQ ID No. 9)

This is an inverse peptide P4+GGGC anchor.

[SEQ ID No. 9]
AQTITEELKAKIGGGC

### Peptide P9 (SEQ ID No. 10)

This is a P4 analogue all amino acids were substituted by most similar ones+GGGC anchor.

[SEQ ID No. 10]
LRVRIDDSLSNVGGGC

### Peptide P10 (SEQ ID No. 11)

This derives from P4, positively-charged side chains A-substituted+GGGC anchor.

[SEQ ID No. 11]
IAAALEETITQAGGGC

### Peptide P11 (SEQ ID No.)

This derives from P4, negatively-charged side chains A-substituted+GGGC anchor.

[SEQ ID No. 12]
IKAKLAATITQAGGGC

### Peptide P12 (SEQ ID No. 13)

This derives from P4, polar side chains A-substituted+GGGC anchor.

[SEQ ID No. 13]
IKAKLEEAIAQAGGGC

Synthetic peptides were covalently coupled to optical sensor chips via the terminal cysteines following the Biacore thiol coupling procedure and SPR binding experiments were run exactly under the same conditions as described in the previous examples. Relative binding strengths of the single peptides were read from three concentration RU400 values and calculated as percentage binding of the peptide P4 reference (P4: RU400=100%). The comparative results of this Example are summarised in Table 1.

TABLE 1
Results of peptide
P4 scanning binding experiments

| | | AA | % BINDING (RU400) | | | |
|---|---|---|---|---|---|---|
| PEPTIDE | DESCRIPTION | SEQUENCE | 0.5 mM | 1 mM | 2 mM | mean |
| P4 | Positive control: | [symbol] | 100 | 100 | 100 | 100 |
| | contains α2δ1 VGGC-α2 | | | | | |
| | C-terminal region | | | | | |
| P6 | C-terminal P4 | [symbol] RYSETLKP [symbol] | 9 | 7 | 9 | 8.3 |
| | extension | | | | | |
| P8 | Inverse peptide P4 | [symbol] | 3 | 2 | 2 | 2.3 |
| P9 | P4 analogue-all aa | LRVRIDDSLSNV [symbol] | 1 | 1 | 2 | 1.3 |
| | substituted by most | | | | | |
| | similar ones | | | | | |
| P10 | P4; positively | IAAA [symbol] | 4 | 4 | 4 | 4 |
| | charged side chains | | | | | |
| | A-substituted | | | | | |
| P11 | P4; negatively | [symbol] AA [symbol] | 23 | 22 | 25 | 23.3 |
| | charged side chains | | | | | |
| | A-substituted | | | | | |
| P12 | P4; polar side | [symbol] AIA [symbol] | 14 | 15 | 17 | 15.3 |
| | chains A-substituted | | | | | |

[symbol]

indicates data missing or illegible when filed

Supported by the lack of binding to the reverse peptide P8, the results of the peptide P4 scanning experiments confirm binding specificity of compound NVA1309 to peptide P4 and indicate that the intact N-terminal amino acid motif IKAKLE is of particular importance to NVA1309 binding.

### Example 7: More Detailed Identification of the Novel Binding Site of Gabapentinoid NVA1309 within the VGCC_α2 Domain of Human α2δ-1 Voltage-Gated Calcium Channel Subunit

In order to identify functional amino acid residues within the binding sequence for NVA1309 within the VGCC_α2 domain of human α2δ-1, the inventors synthesized and used peptides P4 (SEQ ID No. 7), P13 (SEQ ID No. 14), P14 (SEQ ID No. 15) and P15 (SEQ ID No. 16). See below for details.

### Peptide P4

Positive binding reference (100% relative binding): homologous to the carboxy-terminal region of α2δ-1 VGCC-2 domain (MP3)+GGGC anchor.

[SEQ ID No. 7]
IKAKLEETITQAGGGC

### Peptide P13

P4 analogue; K2 A-substituted+GGGC anchor.

[SEQ ID No. 14]
IAAKLEETITQAGGGC

### Peptide P14

P4 analogue; K4 A-substituted+GGGC anchor.

[SEQ ID No. 15]
IKAALEETITQAGGGC

### Peptide P15

amino-terminal part of P4+GGGC anchor.

[SEQ ID No. 16]
IKAKGGGC

Peptide P4 was covalently coupled via the carboxy terminal cysteine to the surface of flow cell 2 (FC2) of a Biacore CM5 optical sensor chip as reference target-ligand using the thiol coupling chemistry. Peptide P13 was covalently thiol-coupled to the surface of flow cell 3 (FC3) of the same sensor chip. Flow cell FC1, representing a blank carboxyl-dextran surface was used as negative (blank) binding reference.

### Binding Analysis of NVA1309 to Peptides P4 and P13

Binding of NVA1309 to peptides P4 and P13 was analysed by generation of concentration dependent sensorgrams. (Background subtracted sensorgrams FC2-FC1 and FC3-FC1)(FIGS. 16A-C).

### Binding Analysis of NVA1309 to Peptides P4 and P14

Binding of NVA1309 to peptides P4 and P14 was analysed by generation of concentration dependent sensorgrams for peptide P14 (see FIG. 17A, background subtracted sensorgrams FC4-FC1). RU400 plots comparing P14 data with the corresponding values obtained for peptide P4 (FC2-FC1; FIG. 16A) is shown in FIG. 17B.

### Binding Analysis of NVA1309 to Peptides P4 and P15

Binding of NVA1309 to peptides P4 and P15 was analysed on a new Biacore CM5 sensor chip by generation of concentration dependent sensorgrams shown below in FIGS. 18A-C (Background subtracted sensorgrams FC4-FC1 and FC3-FC1).

The data illustrated in FIGS. 16 to 18 confirm the specificity of binding of compound NVA1309 to the newly discovered VGCC_α2 target. Using peptide P4 as on-chip reference, relative RU400 values and binding scores presented in Table 2 are consistent and underline the importance of the IKAK sequence and in particular of Lysine (K) in position 4 for compound NVA1309 binding on target. The carboxy-terminal half of P4 (ETITQA) per se was found not to be sufficient to interact with NVA1309.

TABLE 2
Summary of relative peptide binding data
for NVA1309 using P4, P13, P14 and P15

| | | | % BINDING (RU400) | | | | |
|---|---|---|---|---|---|---|---|
| Peptide | Description | AA Sequence | 0.5 mM | 1 mM | 2 mM | Mean | SCORE |
| P4 | Positive control, | IKAKLEETITQAGGGC | 100 | 100 | 100 | 100 | ++++ |
| | contains the α2δ-1 | | | | | | |
| | VGCC_a2 C-Terminal | | | | | | |
| | region | | | | | | |
| P13 | P4; K2 A-substituted | IAAKLEETITQAGGGC | 133 | 88 | 90 | 104 | ++++ |
| P14 | P4; K4 A-substituted | IKAALEETITQAGGGC | 0 | 11 | 6 | 6 | +/− |
| P15 | P4; IKAK core | IKAKGGGC | 19 | 25 | 26 | 23 | ++ |
| | sequence | | | | | | |

### Example 8—Binding of α2δ-1 Recombinant Protein (SEQ ID No. 5) Containing the VGCC_α2 Domain on the Gabapentinoid Compound NVA1309 which is Covalently Immobilized on the Surface of a Biacore CM5 Optical Sensor Chip

In order to confirm data presented in the previous examples and to allow for Biacore SPR experiments with large proteins like recombinant full length α2δ1 constructs (SEQ ID No. 1), an SPR protocol was established which allowed to use compound NVA1309 as ligand by covalently linking the single primary amino group of compound NVA1309 to the carboxyl groups of the dextran matrix as schematically shown in FIG. 19.

Chemical coupling of compound NVA1309 to the sensor chip was achieved by applying compound NVA1309 in a 50 mM stock solution diluted to 2 mM in phosphate buffered saline (PBS) and subsequently following the protocol recommended in the Biacore amine coupling Kit.

The following ligands were coupled to the surface of a Biacore CM5 sensor chip:
Flow cell 1 (FC1): Amine (ethanolamin) activated blank surface
Flow cell 2 (FC2): Non related protein 1 ( . . . RU)
Flow cell 3 (FC3): Non related protein 2 ( . . . RU)
Flow cell 4 (FC4): NVA1309 (44 RU)

α2δ-1 recombinant peptide (101) was passed over all flow cells as analyte. FC1 subtracted sensorgrams are shown in FIG. 20.

In order to estimate the affinity of α2δ-1 recombinant peptide binding to immobilized NVA1309, the binding sensorgram was analyzed by mathematical curve fitting applying a Langmuir 1:1 interaction algorithm (BiaEvaluation 4.1 software) (see FIG. 21).

Kinetics of the α2δ-1 recombinant peptide VGGC_α2-NVA1309 interaction were assessed by determination of kinetic binding constants (k-on; kₐ, k-off; k_{d}) based on curve fitting shown in FIG. 21. The equilibrium dissociation constant K_{D}=k_{d}/kₐ was calculated and provides a single concentration estimate of the compound target binding affinity.

TABLE 3
Kinetic constants and binding affinity of α2δ-1 recombinant peptide
- NVA1309 target interaction (single concentration data)

| compound | kₐ (1/Ms) | k_{d} (1/s) | K_{D} (nM) |
|---|---|---|---|
| NVA1309 | 1.8 × 10⁴ | 6.1 × 10⁻⁴ | 33.8 |

### Example 9—Binding of Human Recombinant Full Length α2δ-1 Protein (SEQ ID No. 1) and Derivatives Thereof (SEQ ID No. 17, SEQ ID No. 18, SEQ ID No. 19) to Compound NVA1309, Covalently Immobilized on the Surface of a Biacore CM5 Optical Sensor Chip

Recombinant full length α2δ-1 protein and mutants containing a single amino acid residue replaced by alanin were generated by standard DNA cloning and expression techniques and used as analytes for SPR binding to amino-coupled compound NVA1309. The analyte proteins shown on Table 4 were investigated:

TABLE 4
Human recombinant full length α2δ-1
proteins and alanine mutants thereof

| human α2δ-1 recombinant protein | Mutant |
|---|---|
| PR1 (SEQ ID No. 1), Construct P1 | wt α2δ-1 |
| PR2 (SEQ ID No. 17), Construct P2 | α2δ-1-R217A |
| PR3 (SEQ ID No. 18), Construct P3 | α2δ-1-K634A |
| PR4 (SEQ ID No. 19), Construct P4 | α2δ-1-R217A + K634A |

PR1 represents native wild type human α2δ-1 protein, PR2 contains a single arginine (R) to alanine (A) substitution described as the binding site for gabapentin/pregabalin within the RRR motif as reported in prior art and literature. PR3 contains a single lysine (K) to alanine (A) substitution representing to the novel binding site for gabapentinoid NVA1309 on the α2δ-1 target, as disclosed in example 7. PR4 protein contains both point mutations as in PR2 and PR3.

Each α2δ-1 construct was run as analyte at three concentrations across the NVA1309 sensor chip surface. (NVA1309 surface density=204RU).

Kinetic binding constants (k-on; kₐ, k-off; k_{d}) were determined from blank amine activated subtracted sensorgrams by mathematical sensorgram fitting. The fitted curves are shown in FIG. 22(A-D).

For calculation of the kinetic constants a Langmuir 1:1 interaction algorithm supplemented with mass transport limitation correction (where applicable) was applied by using the BiaEvaluation 4.1 software. The equilibrium dissociation constant K_{D}=k_{d}/kₐ was calculated. Kinetic binding constants and calculated affinities obtained for all recombinant proteins are summarized in Table 5.

TABLE 5
Kinetic constants and binding affinity of the recombinant
α2δ-1 proteins-NVA1309 target interaction

| human α2δ-1 recombinant | | | | |
|---|---|---|---|---|
| protein | Mutant | Kₐ (1/Ms); e⁻⁴ | K_{d} (1/s); e⁻⁴ | K_{D} (nM) |
| PR1 (Seq. ID NO. 1), | wt α2δ-1 | 4.2 | 1.7 | 3.9 |
| Construct 1 | | | | |
| PR2 (SEQ ID NO. 17), | α2δ-1-R217A | 2.8 | 2.1 | 7.5 |
| Construct 2 | | | | |
| PR3 (SEQ ID NO. 18), | α2δ-1-K634A | 2.5 | 3.1 | 12.4 |
| Construct 3 | | | | |
| PR4 (SEQ ID No. 19), | α2δ-1-R217A + | 0.3 | 2.4 | 83.4 |
| Construct 4 | K634A | | | |

### Conclusion

I-NVA1309 Displays a Novel Mechanism of Interaction with Target

NVA1309 is a gabapentinoid which binds to α2δ-1 by a molecular interaction mechanism different from that of gabapentinoids (Pregabalin/Gabapentin) described in the prior art. NVA1309 was able to bind to both recombinant truncated α2δ-1 proteins whereas no binding signals were obtained for pregabalin. Binding of pregabalin on these two truncated α2δ-1 fragments would have been expected as claimed in prior art and published literature [13]. This surprising finding clearly demonstrates a fundamental difference between compound NVA1309 and pregabalin in respect to their mode of target interaction: It has been shown that intact three dimensional folding of the full length α2δ-1 protein is necessary for pregabalin to interact with its known binding region (RRR motif) as postulated in literature [18], no such structural constraint is mandatory for NVA1309.

This unexpected finding was further extended by showing that compound NVA1309 was also able to bind to a short synthetic peptide exclusively comprising the reported PGB binding region upstream of the VWA domain of α2δ-1 (Peptide P1; Sequence Id 4). In contrast, pregabalin was unable to bind this peptide.

II-NVA1309 Interacts with a Novel Target Region within α2δ-1

Compound NVA1309 was found to independently bind to a region within the VGGC_α2 domain of the α2δ-1 protein, which is located downstream of the reported pregabalin binding sequence. An α2δ-1 recombinant peptide, expressed in yeast and comprising the VGGC_α2 domain but not the known in the prior art pregabalin/gabapentin binding site (RRR) (Sequence Id5), was used for SPR binding experiments.

Unexpectedly, compound NVA1309 was capable to specifically bind to the recombinant α2δ-1 recombinant peptide lacking the α2δ-1 region upstream of the VWF_A domain, in literature referred to as binding site for pregabalin and gabapentin. Binding proved to be specific (Bovine Serum Albumin, BSA as negative binding control) and also was concentration-dependent.

SPR binding analysis using synthetic peptides within the VGGC_α2 binding region were carried out with compound NVA1309 and the specific binding region was further narrowed down to the carboxy-terminal amino acid part of the VGGC_α2 domain. This was achieved by demonstrating NVA1309 binding to a synthetic peptide (peptide P3; SEQ ID No. 6) comprising this carboxy terminal VGGC_α2 region. Additional SPR binding experiments with shorter peptides (peptides P4 (SEQ ID No. 7), and peptide P6 (SEQ ID No. 8) as well as P4 peptide-derivatives containing alanine substituted amino acid residues, P9 (SEQ ID No. 10), peptide P10 (SEQ ID No. 11), peptide P11 (SEQ ID No. and peptide P12 (SEQ ID NO. 13) allowed the identification of a stretch of amino acids comprising the sequence IKAKLEETITQA as a novel binding region involved in target-compound NVA1309 interaction.

Specific interaction of compound NVA1309 with recombinant full length α2δ-1 protein (SEQ ID No. 1) as well as with α2δ-1 recombinant peptide (SEQ ID No. 5) was confirmed by SPR analysis using compound NVA1309 as ligand, after its covalent coupling to optical sensor chip surface via its single primary amino group and by applying the recombinant proteins as analytes in solution. Single recombinant full length α2δ-1 mutated protein containing alanine substitutions for the respective amino acid residues contained within the pregabalin binding site, as described in prior art (SEQ ID No. 14) and/or amino acid residues contained within the newly detected carboxy terminal VGGC_α2 region IKAKLEETITQA (SEQ ID No. 15, SEQ ID No. 16) was also shown to bind to covalently immobilized compound NVA1309.

Determination of kinetic binding constants obtained from the SPR sensorgrams allowed calculation of binding affinities (K_{D} values) of the various analytes to the chemically immobilized compound NVA1309 and were found to lie in the range of 5-80 nM. These novel and unexpected findings postulate a unique mode of target interaction with gabapentinoid compound NVA1309 which is different from the target interaction binding properties of gabapentinoids gabapentin and pregabalin, as reported in the prior art. Furthermore, these findings constitute a novel and inventive basis to be used for the identification of novel voltage-gated calcium channel modulators with potentially unique biological/pharmacological properties.

The results disclosed in Examples 1 to 9, therefore, describe the identification and molecular characterisation of a novel binding site for gabapentinoid compounds within the VGGC_α2 domain of the auxiliary α2δ-1 subunit of voltage-gated calcium (CaV) channels. This target site may operate synergistically with the gabapentin/pregabalin binding site reported in the prior art located upstream of the VWA domain in order to generate a high affinity binding pocket. However, the newly identified novel VGGC_α2-located binding site may also act as stand-alone target for identification and development of novel therapeutically active calcium channel modulators.

### SEQUENCE LISTING

### Peptide Amino Acid Sequences

### SEQ ID NO. 1 (Human Full Length α2δ-1, Numbering Includes the Signal Peptide):

MAAGCLLALTLTLFQSLLIGPSSEEPFPSAVTIKSWVDKMQEDLVTLAKTASGVNQLVDIYEKYQDLY TVEPNNARQLVEIAARDIEKLLSNRSKALVRLALEAEKVQAAHQWREDFASNEVVYYNAKDDLDPEKN DSEPGSQRIKPVFIEDANFGRQISYQHAAVHIPTDIYEGSTIVLNELNWTSALDEVFKKNREEDPSLL WQVFGSATGLARYYPASPWVDNSRTPNKIDLYDVRRRPWYIQGAASPKDMLILVDVSGSVSGLTLKLI RTSVSEMLETLSDDDFVNVASFNSNAQDVSCFQHLVQANVRNKKVLKDAVNNITAKGITDYKKGFSFA FEQLLNYNVSRANCNKIIMLFTDGGEERAQEIFNKYNKDKKVRVFTFSVGQHNYDRGPIQWMACENKG YYYEIPSIGAIRINTQEYLDVLGRPMVLAGDKAKQVQWTNVYLDALELGLVITGTLPVFNITGQFENK TNLKNQLILGVMGVDVSLEDIKRLTPRFTLCPNGYYFAIDPNGYVLLHPNLQPKPIGVGIPTINLRKR RPNIQNPKSQEPVTLDFLDAELENDIKVEIRNKMIDGESGEKTFRTLVKSQDERYIDKGNRTYTWTPV NGTDYSLALVLPTYSFYYIKAKLEETITQARYSETLKPDNFEESGYTFIAPRDYCNDLKISDNNTEFL LNFNEFIDRKTPNNPSCNADLINRVLLDAGFTNELVQNYWSKQKNIKGVKARFVVTDGGITRVYPKEA GENWQENPETYEDSFYKRSLDNDNYVFTAPYFNKSGPGAYESGIMVSKAVEIYIQGKLLKPAVVGIKI DVNSWIENFTKTSIRDPCAGPVCDCKRNSDVMDCVILDDGGFLLMANHDDYTNQIGRFFGEIDPSLMR HLVNISVYAFNKSYDYQSVCEPGAAPKQGAGHRSAYVPSVADILQIGWWATAAAWSILQQFLLSLTFP RLLEAVEMEDDDFTASLSKQSCITEQTQYFFDNDSKSFSGVLDCGNCSRIFHGEKLMNTNLIFIMVES KGTCPCDTRLLIQAEQTSDGPNPCDMVKQPRYRKGPDVCFDNNVLEDYTDCGGVSGLNPSLWYIIGIQ FLLLWLVSGSTHRLL

### SEQ ID NO. 2 (Recombinant α2δ-1 Truncation 1, Construct 1):

ATGLARYYPASPWVDNSRTPNKIDLYDVRRRPWYIQGAASPKGRPMVLAGDKAKQVQWTNVYLDALEL GLVITGTLPVFNITGQFENKTNLKNQLILGVMGVDVSLEDIKRLTPRFTLCPNGYYFAIDPNGYVLLH PNLQPKPIGVGIPTINLRKRRPNIQNPKSQEPVTLDFLDAELENDIKVEIRNKMIDGESGEKTFRTLV KSQDERYIDKGNRTYTWTPVNGTDYSLALVLPTYSFYYIKAKLEETITQARYSET

### SEQ ID No. 3 (Recombinant α2δ-1 Truncation 2, Construct 2):

### ATGLARYYPASPWVDNSRTPNKIDLYDVRRRPWYIQGAASPKGRPMVLAGDKAKQVQKRRPNIQNPKS QEPVTLDFLDAELENDIKVEIRNKMIDGESGEKTFRTLVKSQDERYIDKGNRTYTWTPVNGTDYSLAL VLPTYSFYYIKAKLEETITQARYSET

### SEQ ID NO. 4 (Peptide P1, Containing the Pregabalin Binding Site):

### RTPNKIDLYDVRRRPWYIQGAGGGC

### SEQ ID NO. 5 (Recombinant CACNA2D1 (Cusabio) Fragment):

### QPKPIGVGIPTINLRKRRPNIQNPKSQEPVTLDFLDAELENDIKVEIRNKMIDGESGEKTFRTLVKSQ DERYIDKGNRTYTWTPVNGTDYSLALVLPTYSFYYIKAKLEETITQARYSETLKPDNFEESGYTFIAP RDYCN

### SEQ ID NO. 6 (Peptide P3, Carboxy-Terminal VGGC_α2 Region (I)+GGGC Anchor):

### TYSFYIKAKLEETITQARYSETGGGC

### SEQ ID NO. 7 (Peptide P4, Carboxy-Terminal VGGC_α2 Region (II)+GGGC Anchor):

### IKAKLEETITQAGGGC

### SEQ ID No. 8 (Peptide P6, Carboxy-Terminal VGGC_α2 Region (IV)+GGGC Anchor):

### ETITQARYSETLKPGGGC

### SEQ ID NO. 9 (Peptide P8, Inverse Peptide P4+GGGC Anchor):

### AQTITEELKAKIGGGC

### SEQ ID NO. 10 (Peptide P9, P4 Analogue—all aa Substituted by Most Similar Ones+GGGC Anchor):

### LRVRIDDSLSNVGGGC

### SEQ ID NO. 11 (Peptide P10, P4 Analogue—Positively Charged Side Chains A-Substituted+GGGC Anchor):

### IAAALEETITQAGGGC

### SEQ ID NO. 12 (Peptide P11, P4 Analogue—Negatively Charged Side Chains A-Substituted+GGGC Anchor):

### IKAKLAATITQAGGGC

### SEQ ID NO. 13 (Peptide P12, P4 Analogue—Polar Side Chains A-Substituted+GGGC Anchor):

### IKAKLEEAIAQAGGGC

SEQ ID NO. 14 (Peptide P13, P4 Analogue with Lysine in Position 2 (K) Substituted by Alanine (A)+GGGC Anchor):

### IAAKLEETITQAGGGC

SEQ ID NO. 15 (Peptide P14, P4 Analogue with Lysine in Position 4 (K) Substituted by Alanine (A)+GGGC Anchor):

### IKAALEETITQAGGGC

SEQ ID NO. 16 (Peptide P15, P4 Analogue Containing Only the Amino-Terminal Part of P4+GGGC anchor):

### IKAKGGGC

SEQ ID NO. 17 (Full Length Human α2δ-1 Recombinant Protein PR2 with R217A Mutation):
EPFPSAVTIKSWVDKMQEDLVTLAKTASGVNQLVDIYEKYQDLYTVEPNNARQLVEIAARDIEKLLSNRSKALVR LALEAEKVQAAHQWREDFASNEVVYYNAKDDLDPEKNDSEPGSQRIKPVFIEDANFGRQISYQHAAVHIPTDIYE GSTIVLNELNWTSALDEVFKKNREEDPSLLWQVFGSATGLARYYPASPWVDNSRTPNKIDLYDVRRPWYIQGAA SPKDMLILVDVSGSVSGLTLKLIRTSVSEMLETLSDDDFVNVASFNSNAQDVSCFQHLVQANVRNKKVLKDAVNN ITAKGITDYKKGFSFAFEQLLNYNVSRANCNKIIMLFTDGGEERAQEIFNKYNKDKKVRVFTFSVGQHNYDRGPI QWMACENKGYYYEIPSIGAIRINTQEYLDVLGRPMVLAGDKAKQVQWTNVYLDALELGLVITGTLPVFNITGQFE NKTNLKNQLILGVMGVDVSLEDIKRLTPRFTLCPNGYYFAIDPNGYVLLHPNLQPKPIGVGIPTINLRKRRPNIQ NPKSQEPVTLDFLDAELENDIKVEIRNKMIDGESGEKTFRTLVKSQDERYIDKGNRTYTWTPVNGTDYSLALVLP TYSFYYIKAKLEETITQARYSETLKPDNFEESGYTFIAPRDYCNDLKISDNNTEFLLNFNEFIDRKTPNNPSCNA DLINRVLLDAGFTNELVQNYWSKQKNIKGVKARFVVTDGGITRVYPKEAGENWQENPETYEDSFYKRSLDNDNYV FTAPYFNKSGPGAYESGIMVSKAVEIYIQGKLLKPAVVGIKIDVNSWIENFTKTSIRDPCAGPVCDCKRNSDVMD CVILDDGGFLLMANHDDYTNQIGRFFGEIDPSLMRHLVNISVYAFNKSYDYQSVCEPGAAPKQGAGHRSAYVPSV ADILQIGWWATAAAWSILQQFLLSLTFPRLLEAVEMEDDDFTASLSKQSCITEQTQYFFDNDSKSFSGVLDCGNC SRIFHGEKLMNTNLIFIMVESKGTCPCDTRLLIQAEQTSDGPNPCDMVKQPRYRKGPDVCFDNNVLEDYTDCGGV SGLNPSLWYIIGIQFLLLWLVSGSTHRLL
SEQ ID NO. 18 (Full Length Human α2δ-1 Recombinant Protein PR3 with K634A Mutation):
EPFPSAVTIKSWVDKMQEDLVTLAKTASGVNQLVDIYEKYQDLYTVEPNNARQLVEIAARDIEKLLSNRSKALVR LALEAEKVQAAHQWREDFASNEVVYYNAKDDLDPEKNDSEPGSQRIKPVFIEDANFGRQISYQHAAVHIPTDIYE GSTIVLNELNWTSALDEVFKKNREEDPSLLWQVFGSATGLARYYPASPWVDNSRTPNKIDLYDVRRRPWYIQGAA SPKDMLILVDVSGSVSGLTLKLIRTSVSEMLETLSDDDFVNVASFNSNAQDVSCFQHLVQANVRNKKVLKDAVNN ITAKGITDYKKGFSFAFEQLLNYNVSRANCNKIIMLFTDGGEERAQEIFNKYNKDKKVRVFTFSVGQHNYDRGPI QWMACENKGYYYEIPSIGAIRINTQEYLDVLGRPMVLAGDKAKQVQWTNVYLDALELGLVITGTLPVFNITGQFE NKTNLKNQLILGVMGVDVSLEDIKRLTPRFTLCPNGYYFAIDPNGYVLLHPNLQPKPIGVGIPTINLRKRRPNIQ NPKSQEPVTLDFLDAELENDIKVEIRNKMIDGESGEKTFRTLVKSQDERYIDKGNRTYTWTPVNGTDYSLALVLP TYSFYYIKALEETITQARYSETLKPDNFEESGYTFIAPRDYCNDLKISDNNTEFLLNFNEFIDRKTPNNPSCNA DLINRVLLDAGFTNELVQNYWSKQKNIKGVKARFVVTDGGITRVYPKEAGENWQENPETYEDSFYKRSLDNDNYV FTAPYFNKSGPGAYESGIMVSKAVEIYIQGKLLKPAVVGIKIDVNSWIENFTKTSIRDPCAGPVCDCKRNSDVMD CVILDDGGFLLMANHDDYTNQIGRFFGEIDPSLMRHLVNISVYAFNKSYDYQSVCEPGAAPKQGAGHRSAYVPSV ADILQIGWWATAAAWSILQQFLLSLTFPRLLEAVEMEDDDFTASLSKQSCITEQTQYFFDNDSKSFSGVLDCGNC SRIFHGEKLMNTNLIFIMVESKGTCPCDTRLLIQAEQTSDGPNPCDMVKQPRYRKGPDVCFDNNVLEDYTDCGGV SGLNPSLWYIIGIQFLLLWLVSGSTHRLL
SEQ ID NO. 19 (Full Length Human α2δ-1 Recombinant Protein PR4 with R217A+K634A Mutations)):
EPFPSAVTIKSWVDKMQEDLVTLAKTASGVNQLVDIYEKYQDLYTVEPNNARQLVEIAARDIEKLLSNRSKALVR LALEAEKVQAAHQWREDFASNEVVYYNAKDDLDPEKNDSEPGSQRIKPVFIEDANFGRQISYQHAAVHIPTDIYE GSTIVLNELNWTSALDEVFKKNREEDPSLLWQVFGSATGLARYYPASPWVDNSRTPNKIDLYDVRRAPWYIQGAA SPKDMLILVDVSGSVSGLTLKLIRTSVSEMLETLSDDDFVNVASFNSNAQDVSCFQHLVQANVRNKKVLKDAVNN ITAKGITDYKKGFSFAFEQLLNYNVSRANCNKIIMLFTDGGEERAQEIFNKYNKDKKVRVFTFSVGQHNYDRGPI QWMACENKGYYYEIPSIGAIRINTQEYLDVLGRPMVLAGDKAKQVQWTNVYLDALELGLVITGTLPVFNITGQFE NKTNLKNQLILGVMGVDVSLEDIKRLTPRFTLCPNGYYFAIDPNGYVLLHPNLQPKPIGVGIPTINLRKRRPNIQ NPKSQEPVTLDFLDAELENDIKVEIRNKMIDGESGEKTFRTLVKSQDERYIDKGNRTYTWTPVNGTDYSLALVLP TYSFYYIKAZLEETITQARYSETLKPDNFEESGYTFIAPRDYCNDLKISDNNTEFLLNFNEFIDRKTPNNPSCNA DLINRVLLDAGFTNELVQNYWSKQKNIKGVKARFVVTDGGITRVYPKEAGENWQENPETYEDSFYKRSLDNDNYV FTAPYFNKSGPGAYESGIMVSKAVEIYIQGKLLKPAVVGIKIDVNSWIENFTKTSIRDPCAGPVCDCKRNSDVMD CVILDDGGFLLMANHDDYTNQIGRFFGEIDPSLMRHLVNISVYAFNKSYDYQSVCEPGAAPKQGAGHRSAYVPSV ADILQIGWWATAAAWSILQQFLLSLTFPRLLEAVEMEDDDFTASLSKQSCITEQTQYFFDNDSKSFSGVLDCGNC SRIFHGEKLMNTNLIFIMVESKGTCPCDTRLLIQAEQTSDGPNPCDMVKQPRYRKGPDVCFDNNVLEDYTDCGGV SGLNPSLWYIIGIQFLLLWLVSGSTHRLL

### SEQ ID NO. 20

### IKAK

### SEQ ID NO. 21

### IKAKLE

### SEQ ID NO. 22

### IKAKLEET

### SEQ ID NO. 23

### IKAKLEETITQA

### SEQ ID NO. 24 (PGB Binding Site)

### VRRRPWYIQGAAS

### SEQ ID NO. 25 (RRR Motif)

### RRR

A CRF ASCI text file of the Sequence Listing is submitted with the specification and is herein incorporated by reference; the ACI text file (i) is named SEQLTXT_1_16616752. txt, (ii) was created on 10 May 2021, and (iii) is 47,825 bytes in size.

### NON PATENT CITATIONS

[1]. Dolphin A C (2013) The α2δ subunits of voltage-gated calcium channel Biochim et Biophys Acta 1828: 1541-1549.
[2]. N. Klugbauer, L. Lacinová, E. Marais, M. Hobom, F. Hofmann (1999) Molecular diversity of the calcium channel α2δ subunit, J. Neurosci. 19: 684-691.
[3]. K. A. Wycisk, C. Zeitz, S. Feil, M. Wittmer, U. Forster, J. Neidhardt, B. Wissinger, E. Zrenner, R. Wilke, S. Kohl, W. Berger (2006) Mutation in the auxiliary calcium-channel subunit CACNA2D4 causes autosomal recessive cone dystrophy, Am. J. Hum. Genet. 79: 973-977.
[4]. V. Anantharaman, L. Aravind (2000) Cache—a signalling domain common to animal Ca channel subunits and a class of prokaryotic chemotaxis receptors, Trends Biochem. Sci. 25: 535-537.
[5]. A. C. Dolphin (2012) Calcium channel auxiliary alpha (2) delta and beta subunits: trafficking and one step beyond, Nat. Rev. Neurosci. 13: 542-555.
[6]. C. S. Bauer, M. Nieto-Rostro, W. Rahman, A. Tran-Van-Minh, L. Ferron, L. Douglas, I. Kadurin, Y. Sri Ranjan, L. Fernandez-Alacid, N. S. Millar, A. H. Dickenson, R. Lujan, A. C. Dolphin (2009) The increased trafficking of the calcium channel subunit α2δ-1 to presynaptic terminals in neuropathic pain is inhibited by the α2δ ligand pregabalin, J. Neurosci. 29: 4076-4088.
[7]. Z. D. Luo, S. R. Chaplan, E. S. Higuera, L. S. Sorkin, K. A. Stauderman, M. E. Williams, T. L. Yaksh (2001) Upregulation of dorsal root ganglion α2δ calcium channel subunit and its correlation with allodynia in spinal nerve-injured rats J. Neurosci. 21: 1868-1875.
[8]. C. Y. Li, X. L. Zhang, E. A. Matthews, K. W. Li, A. Kurwa, A. Boroujerdi, J. Gross, M. S. Gold, A. H. Dickenson, G. Feng, Z. D. Luo (2006) Calcium channel alpha (2) delta (1) subunit mediates spinal hyperexcitability in pain modulation, Pain 125: 20-34.
[9]. R. Patel, C S Bauer, M. Nieto-Rostro, W. Margas, L. Ferron, K. Chaggar, K. Crews, J D. Ramirez, DLH Bennett, A. Schwartz, A H Dickenson, and A C Dolphin (2013) α2δ-1 Gene Deletion Affects Somatosensory Neuron Function and Delays Mechanical Hypersensitivity in Response to Peripheral Nerve Damage. J. Neurosci. 33: 16412-16426
[10]. C. P. Taylor, T. Angelotti, E. Fauman (2007) Pharmacology and mechanism of action of pregabalin: the calcium channel alpha2-delta (alpha2-delta) subunit as a target for antiepileptic drug discovery, Epilepsy Res. 73: 137-150.
[11]. N. S. Gee, J. P. Brown, V. U. K. Dissanayake, J. Offord, R. Thurlow, G. N. Woodruff (1996) The novel anticonvulsant drug, gabapentin (Neurontin), binds to the α2δ subunit of a calcium channel, J. Biol. Chem. 271: 5768-5776.
[12]. A. Davies, L. Douglas, J. Hendrich, J. Wratten, A. Tran-Van-Minh, I. Foucault, D. Koch, W. S. Pratt, H. Saibil, A. C. Dolphin (2006) The calcium channel α2δ-2 subunit partitions with CaV2.1 in lipid rafts in cerebellum: implications for localization and function, J. Neurosci. 26: 8748-8757.
[13]. M. J. Field, P. J. Cox, E. Stott, H. Melrose, J. Offord, T. Z. Su, S. Bramwell, L. Corradini, S. England, J. Winks, R. A. Kinloch, J. Hendrich, A. C. Dolphin, T. Webb, D. Williams (2006) Identification of the α2δ-1 subunit of voltage-dependent calcium channels as a novel molecular target for pain mediating the analgesic actions of pregabalin, Proc. Natl. Acad. Sci. U.S.A. 103:17537-17542.
[14]. A. Boroujerdi, J. Zeng, K. Sharp, D. Kim, O. Steward, and Z. D. Luo (2011) Calcium channel alpha-2-delta-1 protein upregulation in dorsal spinal cord mediates spinal cord injury induced neuropathic pain states. Pain 152: 649-655.
[15]. D. Calvo, M J Vázquez, C. Ashby, J M Domínguez (2012) Kinetic considerations on the development of binding assays in single-addition mode: application to the search for α2δ modulators J. Biomol. Screen. 17: 1041-1049.
[16]. W Zhao, L Wang, H Han, K Jin, N Lin, T Guo, Y Chen, H Cheng, F Lu, W Fang, Y Wang, B Xing, and Z Zhang (2013)11B50-1, a mAb Raised against Recurrent Tumor Cells, Targets Liver Tumor-Initiating Cells by Binding to the Calcium Channel a2d-1 Subunit Cancer Cell 23, 541-556.
[17]. ÇEroglu, N J Allen, M W Susman, N A O'Rourke, C Y Park, E Özkan, C Chakraborty, S B. Mulinyawe, D S Annis, A D Huberman, E M Green, J Lawler, R Dolmetsch, K. C Garcia, S J. Smith, Z. D. Luo, A. Rosenthal, D F. Mosher, and B A Barres (2009) The Gabapentin Receptor α2δ-1 is the Neuronal Thrombospondin Receptor Responsible for Excitatory CNS Synaptogenesis Cell 139: 380-392.
[18]. Gee N S, Brown J P, Dissanayake V U K, Offord J, Thurlow R AND Woodruff G N (1996) The novel anticonvulsant drug, gabapentin (Neurontin), binds to the α2δ subunit of a calcium channel. J. Biol. Chem. 271: 5768-5776.

1. An isolated α2δ-1 peptide consisting of a fragment of an amino acid sequence substantially as set out in SEQ ID No. 5 and encompassing an amino acid sequence substantially as set out in SEQ ID No. 20.

2. A peptide according to claim 1, wherein the fragment comprises an amino acid sequence of SEQ ID No. 20, and further includes at least 1, 2, 3, 4, 5, 6, 7, 8, 9 or 10 N-terminal and/or C-terminal amino acids, which correspond to at least 1, 2, 3, 4, 5, 6, 7, 8, 9 or 10 amino acids that are located at the N-terminus and/or C-terminus of the fragment equivalent to SEQ ID No. 20 as disposed within SEQ ID No. 5.

3. A peptide according to claim 1, wherein the peptide comprises an amino acid sequence selected from the group consisting of SEQ ID No. 20, SEQ ID No. 21, SEQ ID No. 22, and SEQ ID No. 23.

4. A peptide according to claim 3, wherein the first four amino acids of SEQ ID Nos. 20 to 23 are not mutated, altered or substituted, preferably, wherein the fourth amino acid of SEQ ID No. 20, SEQ ID No. 21, SEQ ID No. 22 or SEQ ID No. 23 is not mutated, altered or substituted.

5. A peptide according to claim 1, wherein the peptide also encompasses a RRR motif or is conjugated to a separate peptide encompassing a RRR motif.

6. An antibody or antigen-binding fragment thereof capable of binding or interacting with a peptide according to claim 1.

7. An isolated nucleic acid encoding the peptide according to claim 1.

8. The isolated nucleic acid according to claim 7, wherein the nucleic acid encodes an amino acid sequence substantially as set out in SEQ ID No. 20.

9. A genetic construct comprising the nucleic acid according to claim 7.

10. A recombinant vector comprising the genetic construct according to claim 9.

11. A host cell comprising the genetic construct according to claim 9.

12. A method of preparing an isolated α2δ-1 recombinant peptide, the method comprising (i) culturing at least one cell according to claim 11; and (ii) isolating the peptide from the cell to create an isolated α2δ-1 recombinant peptide.

13. A membrane, micelle or liposome comprising the α2δ-1 peptide according to claim 1.

14. A membrane, micelle, liposome or α2δ-1 peptide according to claim 13, wherein the membrane, micelle, liposome or α2δ-1 peptide is recombinant.

15. A membrane, micelle, liposome or α2δ-1 peptide according to claim 14, wherein the membrane is a plasma membrane or an organelle membrane.

16. A binding assay test system for identifying an agent that binds to the α2δ-1 protein of a voltage-gated calcium channel, the system comprising the peptide according to claim 1 or a peptide which comprises an amino acid sequence substantially as set out in SEQ ID No. 20.

17. The test system according to claim 16, wherein the test system comprises a positive control that binds to the isolated peptide.

18. The test system according to claim 16, wherein the test system comprises a negative control that does not bind to the isolated peptide.

19. A method of identifying an agent that binds to the α2δ-1 protein of a voltage-gated calcium channel, the method comprising detecting for binding between the agent and the peptide according to claim 1 or a peptide comprising an amino acid sequence substantially as set out in SEQ ID No. 20.

20. The method according to claim 19, wherein the method comprises using Absorbance, Fluorescence intensity, Luminescence, Surface Plasmon Resonance (SPR), reverse Surface Plasmon Resonance (rSPR), Fluorescence Polarization, Fluorescence resonance energy transfer (FRET), Time resolved Fluorescence (TRF), Homogeneous Time Resolved Fluorescence (HTREF/TR-FRET), Alpha Screen Technology, Fluorescence lifetime, fragment complementation or FLIPR (for calcium readout), ELISA, Radioligand binding assays or Immunoprecipitation.

21. The method according to claim 19, wherein prior to detecting for binding between the agent and the peptide, the method comprises a step of contacting the agent and the peptide according to claim 1 or a peptide comprising an amino acid sequence substantially as set out in SEQ ID No. 20.

22. An agent identified by the method according to claim 19, for use in therapy or as a medicament, or in diagnosis.

23. An agent identified by the method according to claim 19 for use in the treatment of a medical condition in which the α2δ-1 subunit is a therapeutic target, the medical condition being selected from: pain, neuropathic pain, peripheral nervous system pain, central nervous system pain, hyperalgesia, tactile allodynia, fibromyalgia, restless legs syndrome, epilepsy, generalised anxiety disorder, migraine, social phobia, panic disorder, mania, bipolar disorder, and alcohol withdrawal, cancer, urinary tract infections, obstructive pulmonary disease, sexual dysfunction, Kawasaki disease, cardiovascular disorders, (such as angina, heart attacks, heart failure) and respiratory disorders (such as asthma and Chronic Obstructive Pulmonary Disease).

24. A method of treating, preventing or ameliorating a condition in which the α2δ-1 subunit is a therapeutic target, the method comprising administering, to a subject in need of such treatment, a therapeutically effective amount of an agent identified by the method according to claim 19.

25. A pharmaceutical composition of an agent, the composition comprising an agent identified by the method according to claim 19 and a pharmaceutically acceptable vehicle.

26. Use of a peptide comprising an isolated α2δ-1 peptide to identify an agent that binds thereto, wherein the peptide is the peptide according to claim 1 or a peptide comprising an amino acid sequence substantially as set out in SEQ ID No. 20.

27. Use of an isolated α2δ-1 peptide to identify an agent that can be used to treat a medical condition in which the α2δ-1 subunit is a therapeutic target, wherein the peptide is the peptide according to claim 1 or comprises an amino acid sequence substantially as set out in SEQ ID No. 20.

28. The use according to claim 27, wherein the medical condition is selected from: pain, neuropathic pain, peripheral nervous system pain, central nervous system pain, hyperalgesia, tactile allodynia, fibromyalgia, restless legs syndrome, epilepsy, generalised anxiety disorder, migraine, social phobia, panic disorder, mania, bipolar disorder, and alcohol withdrawal, cancer, urinary tract infections, obstructive pulmonary disease, sexual dysfunction, Kawasaki disease, cardiovascular disorders, (such as angina, heart attacks, heart failure) and respiratory disorders (such as asthma and Chronic Obstructive Pulmonary Disease).

29. A membrane, micelle or liposome comprising the α2δ-1 peptide obtained or obtainable by the method according to claim 12.

30. A membrane, micelle, liposome or α2δ-1 peptide according to claim 29, wherein the membrane, micelle, liposome or α2δ-1 peptide is recombinant.

31. A membrane, micelle, liposome or α2δ-1 peptide according to claim 30, wherein the membrane is a plasma membrane or an organelle membrane.


SEQUENCE LISTING (25 sequences)

SEQ ID NO: 1 - protein, 1103 aa, Homo sapiens
MAAGCLLALTLTLFQSLLIGPSSEEPFPSAVTIKSWVDKMQEDLVTLAKTASGVNQLVDIYEKYQDLYTVEPNNARQLVEIAARDIEKLLSNRSKALVRLALEAEKVQAAHQWREDFASNEVVYYNAKDDLDPEKNDSEPGSQRIKPVFIEDANFGRQISYQHAAVHIPTDIYEGSTIVLNELNWTSALDEVFKKNREEDPSLLWQVFGSATGLARYYPASPWVDNSRTPNKIDLYDVRRRPWYIQGAASPKDMLILVDVSGSVSGLTLKLIRTSVSEMLETLSDDDFVNVASFNSNAQDVSCFQHLVQANVRNKKVLKDAVNNITAKGITDYKKGFSFAFEQLLNYNVSRANCNKIIMLFTDGGEERAQEIFNKYNKDKKVRVFTFSVGQHNYDRGPIQWMACENKGYYYEIPSIGAIRINTQEYLDVLGRPMVLAGDKAKQVQWTNVYLDALELGLVITGTLPVFNITGQFENKTNLKNQLILGVMGVDVSLEDIKRLTPRFTLCPNGYYFAIDPNGYVLLHPNLQPKPIGVGIPTINLRKRRPNIQNPKSQEPVTLDFLDAELENDIKVEIRNKMIDGESGEKTFRTLVKSQDERYIDKGNRTYTWTPVNGTDYSLALVLPTYSFYYIKAKLEETITQARYSETLKPDNFEESGYTFIAPRDYCNDLKISDNNTEFLLNFNEFIDRKTPNNPSCNADLINRVLLDAGFTNELVQNYWSKQKNIKGVKARFVVTDGGITRVYPKEAGENWQENPETYEDSFYKRSLDNDNYVFTAPYFNKSGPGAYESGIMVSKAVEIYIQGKLLKPAVVGIKIDVNSWIENFTKTSIRDPCAGPVCDCKRNSDVMDCVILDDGGFLLMANHDDYTNQIGRFFGEIDPSLMRHLVNISVYAFNKSYDYQSVCEPGAAPKQGAGHRSAYVPSVADILQIGWWATAAAWSILQQFLLSLTFPRLLEAVEMEDDDFTASLSKQSCITEQTQYFFDNDSKSFSGVLDCGNCSRIFHGEKLMNTNLIFIMVESKGTCPCDTRLLIQAEQTSDGPNPCDMVKQPRYRKGPDVCFDNNVLEDYTDCGGVSGLNPSLWYIIGIQFLLLWLVSGSTHRLL

SEQ ID NO: 2 - protein, 259 aa, Artificial Sequence
  recombinant a2d-1 truncation 1, Construct 1
ATGLARYYPASPWVDNSRTPNKIDLYDVRRRPWYIQGAASPKGRPMVLAGDKAKQVQWTNVYLDALELGLVITGTLPVFNITGQFENKTNLKNQLILGVMGVDVSLEDIKRLTPRFTLCPNGYYFAIDPNGYVLLHPNLQPKPIGVGIPTINLRKRRPNIQNPKSQEPVTLDFLDAELENDIKVEIRNKMIDGESGEKTFRTLVKSQDERYIDKGNRTYTWTPVNGTDYSLALVLPTYSFYYIKAKLEETITQARYSET

SEQ ID NO: 3 - protein, 162 aa, Artificial Sequence
  recombinant a2d-1 truncation 2, Construct 2
ATGLARYYPASPWVDNSRTPNKIDLYDVRRRPWYIQGAASPKGRPMVLAGDKAKQVQKRRPNIQNPKSQEPVTLDFLDAELENDIKVEIRNKMIDGESGEKTFRTLVKSQDERYIDKGNRTYTWTPVNGTDYSLALVLPTYSFYYIKAKLEETITQARYSET

SEQ ID NO: 4 - protein, 25 aa, Artificial Sequence
  Peptide P1, containing the Pregabalin binding site
RTPNKIDLYDVRRRPWYIQGAGGGC

SEQ ID NO: 5 - protein, 141 aa, Artificial Sequence
  recombinant CACNA2D1 (Cusabio) fragment
QPKPIGVGIPTINLRKRRPNIQNPKSQEPVTLDFLDAELENDIKVEIRNKMIDGESGEKTFRTLVKSQDERYIDKGNRTYTWTPVNGTDYSLALVLPTYSFYYIKAKLEETITQARYSETLKPDNFEESGYTFIAPRDYCN

SEQ ID NO: 6 - protein, 26 aa, Artificial Sequence
  Peptide P3, carboxy-terminal VGGC_a2 region (I) + GGGC anchor
TYSFYIKAKLEETITQARYSETGGGC

SEQ ID NO: 7 - protein, 16 aa, Artificial Sequence
  Peptide P4, carboxy-terminal VGGC_a2 region (II) + GGGC anchor
IKAKLEETITQAGGGC

SEQ ID NO: 8 - protein, 18 aa, Artificial Sequence
  Peptide P6, carboxy-terminal VGGC_a2 region (IV) + GGGC anchor
ETITQARYSETLKPGGGC

SEQ ID NO: 9 - protein, 16 aa, Artificial Sequence
  Peptide P8,  Inverse peptide P4 + GGGC anchor
AQTITEELKAKIGGGC

SEQ ID NO: 10 - protein, 16 aa, Artificial Sequence
  Peptide P9, P4 analogue - all aa substituted by most similar ones + GGGC anchor
LRVRIDDSLSNVGGGC

SEQ ID NO: 11 - protein, 16 aa, Artificial Sequence
  Peptide P10, P4 analogue - positively charged side chains A-substituted + GGGC anchor
IAAALEETITQAGGGC

SEQ ID NO: 12 - protein, 16 aa, Artificial Sequence
  Peptide P11, P4 analogue -  negatively charged side chains A-substituted + GGGC anchor
IKAKLAATITQAGGGC

SEQ ID NO: 13 - protein, 16 aa, Artificial Sequence
  Peptide P12, P4 analogue -  polar side chains A-substituted + GGGC anchor
IKAKLEEAIAQAGGGC

SEQ ID NO: 14 - protein, 16 aa, Artificial Sequence
  Peptide P13, P4 analogue with Lysine in position 2 (K) substituted by Alanine (A) + GGGC anchor
IAAKLEETITQAGGGC

SEQ ID NO: 15 - protein, 16 aa, Artificial Sequence
  Peptide P14, P4 analogue with Lysine in position 4 (K) substituted by Alanine (A) + GGGC anchor
IKAALEETITQAGGGC

SEQ ID NO: 16 - protein, 8 aa, Artificial Sequence
  Peptide P15, P4 analogue containing only the amino-terminal part of P4 + GGGC anchor
IKAKGGGC

SEQ ID NO: 17 - protein, 1079 aa, Homo sapiens
EPFPSAVTIKSWVDKMQEDLVTLAKTASGVNQLVDIYEKYQDLYTVEPNNARQLVEIAARDIEKLLSNRSKALVRLALEAEKVQAAHQWREDFASNEVVYYNAKDDLDPEKNDSEPGSQRIKPVFIEDANFGRQISYQHAAVHIPTDIYEGSTIVLNELNWTSALDEVFKKNREEDPSLLWQVFGSATGLARYYPASPWVDNSRTPNKIDLYDVRRAPWYIQGAASPKDMLILVDVSGSVSGLTLKLIRTSVSEMLETLSDDDFVNVASFNSNAQDVSCFQHLVQANVRNKKVLKDAVNNITAKGITDYKKGFSFAFEQLLNYNVSRANCNKIIMLFTDGGEERAQEIFNKYNKDKKVRVFTFSVGQHNYDRGPIQWMACENKGYYYEIPSIGAIRINTQEYLDVLGRPMVLAGDKAKQVQWTNVYLDALELGLVITGTLPVFNITGQFENKTNLKNQLILGVMGVDVSLEDIKRLTPRFTLCPNGYYFAIDPNGYVLLHPNLQPKPIGVGIPTINLRKRRPNIQNPKSQEPVTLDFLDAELENDIKVEIRNKMIDGESGEKTFRTLVKSQDERYIDKGNRTYTWTPVNGTDYSLALVLPTYSFYYIKAKLEETITQARYSETLKPDNFEESGYTFIAPRDYCNDLKISDNNTEFLLNFNEFIDRKTPNNPSCNADLINRVLLDAGFTNELVQNYWSKQKNIKGVKARFVVTDGGITRVYPKEAGENWQENPETYEDSFYKRSLDNDNYVFTAPYFNKSGPGAYESGIMVSKAVEIYIQGKLLKPAVVGIKIDVNSWIENFTKTSIRDPCAGPVCDCKRNSDVMDCVILDDGGFLLMANHDDYTNQIGRFFGEIDPSLMRHLVNISVYAFNKSYDYQSVCEPGAAPKQGAGHRSAYVPSVADILQIGWWATAAAWSILQQFLLSLTFPRLLEAVEMEDDDFTASLSKQSCITEQTQYFFDNDSKSFSGVLDCGNCSRIFHGEKLMNTNLIFIMVESKGTCPCDTRLLIQAEQTSDGPNPCDMVKQPRYRKGPDVCFDNNVLEDYTDCGGVSGLNPSLWYIIGIQFLLLWLVSGSTHRLL

SEQ ID NO: 18 - protein, 1079 aa, Homo sapiens
EPFPSAVTIKSWVDKMQEDLVTLAKTASGVNQLVDIYEKYQDLYTVEPNNARQLVEIAARDIEKLLSNRSKALVRLALEAEKVQAAHQWREDFASNEVVYYNAKDDLDPEKNDSEPGSQRIKPVFIEDANFGRQISYQHAAVHIPTDIYEGSTIVLNELNWTSALDEVFKKNREEDPSLLWQVFGSATGLARYYPASPWVDNSRTPNKIDLYDVRRRPWYIQGAASPKDMLILVDVSGSVSGLTLKLIRTSVSEMLETLSDDDFVNVASFNSNAQDVSCFQHLVQANVRNKKVLKDAVNNITAKGITDYKKGFSFAFEQLLNYNVSRANCNKIIMLFTDGGEERAQEIFNKYNKDKKVRVFTFSVGQHNYDRGPIQWMACENKGYYYEIPSIGAIRINTQEYLDVLGRPMVLAGDKAKQVQWTNVYLDALELGLVITGTLPVFNITGQFENKTNLKNQLILGVMGVDVSLEDIKRLTPRFTLCPNGYYFAIDPNGYVLLHPNLQPKPIGVGIPTINLRKRRPNIQNPKSQEPVTLDFLDAELENDIKVEIRNKMIDGESGEKTFRTLVKSQDERYIDKGNRTYTWTPVNGTDYSLALVLPTYSFYYIKAALEETITQARYSETLKPDNFEESGYTFIAPRDYCNDLKISDNNTEFLLNFNEFIDRKTPNNPSCNADLINRVLLDAGFTNELVQNYWSKQKNIKGVKARFVVTDGGITRVYPKEAGENWQENPETYEDSFYKRSLDNDNYVFTAPYFNKSGPGAYESGIMVSKAVEIYIQGKLLKPAVVGIKIDVNSWIENFTKTSIRDPCAGPVCDCKRNSDVMDCVILDDGGFLLMANHDDYTNQIGRFFGEIDPSLMRHLVNISVYAFNKSYDYQSVCEPGAAPKQGAGHRSAYVPSVADILQIGWWATAAAWSILQQFLLSLTFPRLLEAVEMEDDDFTASLSKQSCITEQTQYFFDNDSKSFSGVLDCGNCSRIFHGEKLMNTNLIFIMVESKGTCPCDTRLLIQAEQTSDGPNPCDMVKQPRYRKGPDVCFDNNVLEDYTDCGGVSGLNPSLWYIIGIQFLLLWLVSGSTHRLL

SEQ ID NO: 19 - protein, 1079 aa, Homo sapiens
EPFPSAVTIKSWVDKMQEDLVTLAKTASGVNQLVDIYEKYQDLYTVEPNNARQLVEIAARDIEKLLSNRSKALVRLALEAEKVQAAHQWREDFASNEVVYYNAKDDLDPEKNDSEPGSQRIKPVFIEDANFGRQISYQHAAVHIPTDIYEGSTIVLNELNWTSALDEVFKKNREEDPSLLWQVFGSATGLARYYPASPWVDNSRTPNKIDLYDVRRAPWYIQGAASPKDMLILVDVSGSVSGLTLKLIRTSVSEMLETLSDDDFVNVASFNSNAQDVSCFQHLVQANVRNKKVLKDAVNNITAKGITDYKKGFSFAFEQLLNYNVSRANCNKIIMLFTDGGEERAQEIFNKYNKDKKVRVFTFSVGQHNYDRGPIQWMACENKGYYYEIPSIGAIRINTQEYLDVLGRPMVLAGDKAKQVQWTNVYLDALELGLVITGTLPVFNITGQFENKTNLKNQLILGVMGVDVSLEDIKRLTPRFTLCPNGYYFAIDPNGYVLLHPNLQPKPIGVGIPTINLRKRRPNIQNPKSQEPVTLDFLDAELENDIKVEIRNKMIDGESGEKTFRTLVKSQDERYIDKGNRTYTWTPVNGTDYSLALVLPTYSFYYIKAALEETITQARYSETLKPDNFEESGYTFIAPRDYCNDLKISDNNTEFLLNFNEFIDRKTPNNPSCNADLINRVLLDAGFTNELVQNYWSKQKNIKGVKARFVVTDGGITRVYPKEAGENWQENPETYEDSFYKRSLDNDNYVFTAPYFNKSGPGAYESGIMVSKAVEIYIQGKLLKPAVVGIKIDVNSWIENFTKTSIRDPCAGPVCDCKRNSDVMDCVILDDGGFLLMANHDDYTNQIGRFFGEIDPSLMRHLVNISVYAFNKSYDYQSVCEPGAAPKQGAGHRSAYVPSVADILQIGWWATAAAWSILQQFLLSLTFPRLLEAVEMEDDDFTASLSKQSCITEQTQYFFDNDSKSFSGVLDCGNCSRIFHGEKLMNTNLIFIMVESKGTCPCDTRLLIQAEQTSDGPNPCDMVKQPRYRKGPDVCFDNNVLEDYTDCGGVSGLNPSLWYIIGIQFLLLWLVSGSTHRLL

SEQ ID NO: 20 - protein, 4 aa, Artificial Sequence
  amino acid sequence
IKAK

SEQ ID NO: 21 - protein, 6 aa, Artificial Sequence
  amino acid sequence
IKAKLE

SEQ ID NO: 22 - protein, 8 aa, Artificial Sequence
  amino acid sequence
IKAKLEET

SEQ ID NO: 23 - protein, 12 aa, Artificial Sequence
  amino acid sequence
IKAKLEETITQA

SEQ ID NO: 24 - protein, 13 aa, Artificial Sequence
  PGB binding site
VRRRPWYIQGAAS

SEQ ID NO: 25 - protein, 3 aa, Artificial Sequence
  RRR motif
RRR